SEQUENCE LISTING (63 sequences)

SEQ ID NO: 1 - protein, 11 aa, synthetic construct
GYSITSDYAWN

SEQ ID NO: 2 - protein, 16 aa, synthetic construct
YLSYSGVTSYNPSLKG

SEQ ID NO: 3 - protein, 16 aa, synthetic construct
KGTYYRYEGSYWYFDV

SEQ ID NO: 4 - protein, 11 aa, synthetic construct
HANQNIDVWLS

SEQ ID NO: 5 - protein, 7 aa, synthetic construct
KTSNLHT

SEQ ID NO: 6 - protein, 9 aa, synthetic construct
QQGQSYPLT

SEQ ID NO: 7 - protein, 125 aa, synthetic construct
QVQLQESGPGLVKPSQTLSLTCTVSGYSITSDYAWNWIRQHPGKGLEWIGYLSYSGVTSYNPSLKGRVTISVDTSKNQFSLKLSSVTAADTAVYYCARKGTYYRYEGSYWYFDVWGQGTTVTVSS

SEQ ID NO: 8 - protein, 125 aa, synthetic construct
QVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSS

SEQ ID NO: 9 - protein, 125 aa, synthetic construct
QVQLQESGPGLVKPSQTLSLTCTVSGYSITSDYAWNWIRQHPGKGLQWMGYLSYSGVTSYNPSLKGRITISRDTSKNQFSLKLSSVTAADTAVYYCARKGTYYRYEGSYWYFDVWGQGTTVTVSS

SEQ ID NO: 10 - protein, 125 aa, synthetic construct
QVQLQESGPGLVKPSQTLSLTCTVSGYSITSDYAWNWIRQHPGKGLQWMGYLSYSGVTSYNPSLKGRITISRDTSKNQFFLKLSSVTAADTAVYYCARKGTYYRYEGSYWYFDVWGQGTTVTVSS

SEQ ID NO: 11 - protein, 107 aa, synthetic construct
DIQMTQSPSSLSASVGDRVTITCHANQNIDVWLSWYQQKPGKAPKLLIYKTSNLHTGVPSRFSGSGSGTDFTFTISSLQPEDIATYYCQQGQSYPLTFGGGTKLEIK

SEQ ID NO: 12 - protein, 107 aa, synthetic construct
DIQMTQSPSSLSASVGDRVTITCHANQNIDVWLSWYQQKPGKAPKLLIYKTSNLHTGVPSRFSGSGSGTDFTLTISSLQPEDIATYYCQQGQSYPLTFGGGTKLEIK

SEQ ID NO: 13 - protein, 107 aa, synthetic construct
DIQMTQSPSSLSASVGDRVTITCHANQNIDVWLSWYQQKPGNAPKLLIYKTSNLHTGVPSRFSGSGSGTDFTFTISSLQPEDIATYYCQQGQSYPLTFGGGTKLEIK

SEQ ID NO: 14 - protein, 107 aa, synthetic construct
DIQMTQSPSSLSASVGDRVTITCHANQNIDVWLSWYQQKPGNAPKLLIYKTSNLHTGVPSRFSGSGSGTDFTLTISSLQPEDIATYYCQQGQSYPLTFGGGTKLEIK

SEQ ID NO: 15 - protein, 21 aa, synthetic construct
RDSDSNCSSEPGFWAHPPGAQ

SEQ ID NO: 16 - protein, 4 aa, synthetic construct
EPGF

SEQ ID NO: 17 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagccttctcagaccctgagcctcacctgtaccgtgtccggatatagcatcacaagcgactacgcctggaattggatcagacagcacccaggcaagggcctggaatggatcggctacctgtcctacagcggcgtgacatcttataaccccagcctgaaaggaagagtgaccatcagcgtggacacctctaagaaccagttcagcctgaagctgtctagcgtcacagccgctgataccgccgtgtactactgcgccagaaagggcacctactaccggtacgagggcagctactggtacttcgacgtgtggggccagggcacaaccgtgaccgtttccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 18 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 19 - DNA, 1428 bp, synthetic construct
  source 1..1428: mol_type = other DNA
atgggctggtcatgcattattctgtttctggtcgcaactgctacaggcgtgcatagtcaagtgcagctgcaggagtctggacctggactggtgaagcctagccagaccctgagcctgacctgtaccgtctccggctacagcatcacctccgactacgcctggaattggatcagacagcaccccggcaagggcctccagtggatgggctacctgtcttattctggagtgacatcttacaacccaagcctgaaaggcagaatcaccatcagccgggacaccagcaagaaccagttcagcctgaagctgagcagcgtgaccgccgctgatacagccgtgtactactgcgccagaaagggcacctactaccggtacgaaggcagctattggtacttcgacgtgtggggccagggcacaacagtgaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 20 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagcctagccagaccctgtccctgacatgtaccgtgtctggctacagcatcacaagcgactacgcctggaattggatccggcagcacccaggaaagggcctccagtggatgggctatctgagctatagcggcgtgacatcctacaaccccagcctgaaaggaagaatcaccatcagcagagatacatctaagaaccagtttttcctgaagctgtctagcgtgaccgccgctgacaccgccgtgtactactgcgccagaaagggcacctactaccggtacgaaggcagctactggtacttcgacgtgtggggccagggcacaaccgtcaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 21 - DNA, 705 bp, synthetic construct
  source 1..705: mol_type = other DNA
atgggctggtcatgtattattctgtttctggtcgcaactgctacaggggtccatagtgatattcagatgacccagagccccagcagcctctccgccagcgtgggagatagagtgaccatcacctgtcacgccaaccagaatatcgacgtctggctgagctggtatcagcagaaacctggcaaggcccctaagctgctgatctacaagaccagcaacctgcacaccggcgtgccatctcggttcagcggctctggcagcggcaccgacttcacatttacaatcagctctctgcagcctgaggacatcgctacatactactgccagcaaggccagtcctaccccctgaccttcggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 22 - DNA, 705 bp, synthetic construct
  source 1..705: mol_type = other DNA
atgggctggtcatgtattattctgtttctggtcgcaactgctacaggggtccatagtgatattcagatgacccagtccccaagcagcctctccgccagcgtgggcgatagagtgaccatcacctgtcacgccaaccagaatatcgacgtgtggctgagctggtaccagcaaaagcccggcaaagcccctaagctgctgatctacaagaccagcaacctgcacaccggcgtccccagccggttcagcggatctggcagcggcaccgacttcaccctgacaatcagctctctgcagcctgaggacatcgctacatactactgccagcagggccagtcttatcctctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 23 - DNA, 705 bp, synthetic construct
  source 1..705: mol_type = other DNA
atgggctggtcatgtattattctgtttctggtcgcaactgctacaggggtccatagtgatattcagatgacccagagccccagcagcctgtccgccagcgtcggcgatagagtgacaatcacctgtcacgccaaccagaatatcgacgtgtggctgtcttggtatcagcaaaaacctggcaacgctcctaagctcctgatctacaagaccagcaacctgcacacaggcgtgccaagccggttcagcggcagcggatctggcaccgacttcaccttcacaatcagctctctgcagcctgaggacatcgccacctactactgccagcagggccagtcctaccccctgacctttggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 24 - DNA, 705 bp, synthetic construct
  source 1..705: mol_type = other DNA
atgggctggtcatgtattattctgtttctggtcgcaactgctacaggggtccatagtgatattcagatgacccagtctccatctagcctctccgccagcgtgggcgaccgggtgaccatcacctgtcacgccaaccagaacatcgacgtgtggctgagctggtatcagcagaaacctggaaatgcccctaagctgctgatctacaagaccagcaacctgcacaccggcgtccccagcagattcagcggcagcggctctggcaccgacttcaccctgacaatcagcagcctgcagcctgaggatatcgctacatactactgccagcaaggccagtcctaccccctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 25 - protein, 125 aa, synthetic construct
QVQLQESGPGLVKPSQSLSLTCTVTGYSITSDYAWNWIRQFPGNKLQWMGYLSYSGVTSYNPSLKGRISITRDTSKNQFFLQLSSVTPEDTATYYCARKGTYYRYEGSYWYFDVWGAGTTVTVSS

SEQ ID NO: 26 - protein, 107 aa, synthetic construct
DIQMNQSPSSLSASLGDTITITCHANQNIDVWLSWYQQKPGNVPKLLIYKTSNLHTGVPSRFSGSGSGTDFTLTISSLQPEDIATYYCQQGQSYPLTFGGGTKLEIK

SEQ ID NO: 27 - DNA, 1428 bp, synthetic construct
  source 1..1428: mol_type = other DNA
atgggctggtcatgcattattctgtttctggtcgcaactgctacaggcgtgcatagtcaggtgcagctgcaggagagcggacctggcctggtgaagcctagccagagcctgtccctcacctgtaccgtgaccggctactccatcacaagcgactacgcctggaattggatcagacagtttccaggcaacaagctgcagtggatgggctacctgtcttattctggcgtgacatcttacaaccccagcctgaaaggcagaatcagcatcacccgggacaccagcaagaaccagttcttcctgcaactgagcagcgtgacccctgaagataccgccacatactactgcgccagaaagggcacctactaccggtacgagggcagctattggtacttcgacgtgtggggcgctggaacaaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 28 - DNA, 705 bp, synthetic construct
  source 1..705: mol_type = other DNA
atgggctggtcatgtattattctgtttctggtcgcaactgctacaggggtccatagtgatatccagatgaaccagagcccttctagcctctccgccagcctgggcgacaccattacaatcacctgtcacgctaatcagaacatcgacgtgtggctgagctggtatcagcagaaacctggcaacgtgcccaagctgctgatctacaagaccagcaacctgcacaccggcgtgccatctagattcagcggatctggcagcggcaccgacttcaccctgaccatcagcagcctgcagcctgaggatatcgccacatactactgccagcaaggccagtcctaccccctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 29 - protein, 25 aa, synthetic construct
QVQLQESGPGLVKPSQSLSLTCTVT

SEQ ID NO: 30 - protein, 14 aa, synthetic construct
WIRQFPGNKLQWMG

SEQ ID NO: 31 - protein, 32 aa, synthetic construct
RISITRDTSKNQFFLQLSSVTPEDTATYYCAR

SEQ ID NO: 32 - protein, 11 aa, synthetic construct
WGAGTTVTVSS

SEQ ID NO: 33 - protein, 25 aa, synthetic construct
QVQLQESGPGLVKPSQTLSLTCTVS

SEQ ID NO: 34 - protein, 14 aa, synthetic construct
WIRQHPGKGLEWIG

SEQ ID NO: 35 - protein, 32 aa, synthetic construct
RVTISVDTSKNQFSLKLSSVTAADTAVYYCAR

SEQ ID NO: 36 - protein, 11 aa, synthetic construct
WGQGTTVTVSS

SEQ ID NO: 37 - protein, 14 aa, unidentified
WIRQHPGKGLEWMG

SEQ ID NO: 38 - protein, 32 aa, synthetic construct
RVTISRDTSKNQFSLKLSSVTAADTAVYYCAR

SEQ ID NO: 39 - protein, 14 aa, synthetic construct
WIRQHPGKGLQWMG

SEQ ID NO: 40 - protein, 32 aa, synthetic construct
RITISRDTSKNQFSLKLSSVTAADTAVYYCAR

SEQ ID NO: 41 - protein, 32 aa, synthetic construct
RITISRDTSKNQFFLKLSSVTAADTAVYYCAR

SEQ ID NO: 42 - protein, 23 aa, synthetic construct
DIQMNQSPSSLSASLGDTITITC

SEQ ID NO: 43 - protein, 15 aa, synthetic construct
WYQQKPGNVPKLLIY

SEQ ID NO: 44 - protein, 32 aa, synthetic construct
GVPSRFSGSGSGTDFTLTISSLQPEDIATYYC

SEQ ID NO: 45 - protein, 10 aa, synthetic construct
FGGGTKLEIK

SEQ ID NO: 46 - protein, 23 aa, synthetic construct
DIQMTQSPSSLSASVGDRVTITC

SEQ ID NO: 47 - protein, 15 aa, synthetic construct
WYQQKPGKAPKLLIY

SEQ ID NO: 48 - protein, 32 aa, synthetic construct
GVPSRFSGSGSGTDFTFTISSLQPEDIATYYC

SEQ ID NO: 49 - protein, 15 aa, synthetic construct
WYQQKPGNAPKLLIY

SEQ ID NO: 50 - protein, 474 aa, synthetic construct
MGWSCIILFLVATATGVHSQVQLQESGPGLVKPSQTLSLTCTVSGYSITSDYAWNWIRQHPGKGLEWIGYLSYSGVTSYNPSLKGRVTISVDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 474 aa, synthetic construct
MGWSCIILFLVATATGVHSQVQLQESGPGLVKPSQTLSLTCTVSGYSITSDYAWNWIRQHPGKGLQWMGYLSYSGVTSYNPSLKGRITISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 474 aa, synthetic construct
MGWSCIILFLVATATGVHSQVQLQESGPGLVKPSQTLSLTCTVSGYSITSDYAWNWIRQHPGKGLQWMGYLSYSGVTSYNPSLKGRITISRDTSKNQFFLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 471 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 233 aa, synthetic construct
MGWSCIILFLVATATGVHSDIQMTQSPSSLSASVGDRVTITCHANQNIDVWLSWYQQKPGKAPKLLIYKTSNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 233 aa, synthetic construct
MGWSCIILFLVATATGVHSDIQMTQSPSSLSASVGDRVTITCHANQNIDVWLSWYQQKPGKAPKLLIYKTSNLHTGVPSRFSGSGSGTDFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 233 aa, synthetic construct
MGWSCIILFLVATATGVHSDIQMTQSPSSLSASVGDRVTITCHANQNIDVWLSWYQQKPGNAPKLLIYKTSNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 233 aa, synthetic construct
MGWSCIILFLVATATGVHSDIQMTQSPSSLSASVGDRVTITCHANQNIDVWLSWYQQKPGNAPKLLIYKTSNLHTGVPSRFSGSGSGTDFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 1425 bp, synthetic construct
  source 1..1425: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcacaaagggccctagcgtgtttccactggccccctcctctaagtccacctctggaggaacagccgccctgggctgtctggtgaaggattatttcccagagcccgtgaccgtgtcctggaactctggcgccctgaccagcggagtgcacacatttccagccgtgctgcagagctccggactgtactccctgtctagcgtggtgaccgtgccttcctctagcctgggcacccagacatatatctgcaacgtgaatcacaagccctccaatacaaaggtggacaagaaggtggagcctaagtcttgtgataagacccacacatgccccccttgtcctgcaccagaggcagcaggaggaccttccgtgttcctgtttccacccaagccaaaggacaccctgatgatcagccgcacccctgaggtgacatgcgtggtggtggacgtgtcccacgaggatccagaggtgaagtttaactggtacgtggatggcgtggaggtgcacaatgccaagaccaagcctcgggaggagcagtacaactctacctatagagtggtgagcgtgctgacagtgctgcaccaggactggctgaacggcaaggagtataagtgcaaggtgtctaataaggccctgcccgcccctatcgagaagaccatcagcaaggcaaagggacagcctagggagccacaggtgtacacactgcctccatctagagacgagctgaccaagaaccaggtgagcctgacatgtctggtgaagggcttctatccaagcgatatcgccgtggagtgggagtccaatggccagcccgagaacaattacaagaccacaccccctgtgctggacagcgatggctccttctttctgtattccaagctgaccgtggataagtctcggtggcagcagggcaacgtgtttagctgttccgtgatgcacgaggccctgcacaatcactacacccagaagtctctgagcctgtcccccggcaagtga

SEQ ID NO: 62 - DNA, 1425 bp, synthetic construct
  source 1..1425: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacgcctctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 63 - DNA, 1416 bp, synthetic construct
  source 1..1416: mol_type = other DNA
atgggctggtcttgtattattctgtttctggtcgcaactgctacaggcgtgcattctcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaagtga

(19) United States
(12) Patent Application Publication
(10) Pub. No.: US 2026/0209321 A1
(43) Pub. Date: Jul. 23, 2026

(54) AN ANTI-TRPM4 ANTIBODY FOR TREATING CYTOKINE-ASSOCIATED VASCULAR DISEASES

(71) Applicant: SINGAPORE HEALTH SERVICES PTE LTD, Singapore (SG)

(72) Inventor: PING Liao, Singapore (SG)

(73) Assignee: SINGAPORE HEALTH SERVICES PTE LTD, Singapore (SG)

(21) Appl. No.: 19/138,017

(22) PCT Filed: Dec. 11, 2023
(86) PCT No.: PCT/SG2023/050822

§ 371 Date: Jun. 11, 2025

(30) Foreign Application Priority Data
Dec. 12, 2022 (SG) 10202260417W

(51) Int. Cl. C07K 16/18; A61P 11/00; A61P 37/06

(52) U.S. Cl. CPC C07K 16/18; A61P 11/00; A61P 37/06; C07K 2317/24

(57) ABSTRACT

Disclosed are anti-transient receptor potential melastatin 4 (TRPM4) antibodies and their use for treating cytokine-associated vascular diseases. In particular, disclosed are humanized monoclonal antibodies specific to TRPM4 and their use for treating cytokine-associated vascular diseases, including cytokine-associated pulmonary injury and cytokine-associated pulmonary vascular damage.

### CROSS-REFERENCE TO RELATED APPLICATION

This application claims the benefit of priority of Singapore Application No. 10202260417W, filed 12 Dec. 2022, the contents of it being hereby incorporated by reference in its entirety for all purposes.

### FIELD OF INVENTION

The present disclosure relates to anti-transient receptor potential melastatin 4 (TRPM4) antibodies and their use for treating cytokine-associated vascular diseases. In particular, the present disclosure relates to humanized monoclonal antibodies specific to TRPM4 and their use for treating cytokine-associated vascular diseases, including cytokine-associated pulmonary injury and cytokine-associated pulmonary vascular damage.

### BACKGROUND

TRPM4 is a nonselective cation channel, permeable to monovalent cation channels. Once upregulated and activated under hypoxia or stress conditions, excessive Na⁺ entry via TRPM4 channel could lead to oncotic cell death. Importantly, under inflammation and hypoxia conditions, TRPM4 activity is enhanced, and blocking TRPM4 could salvage affected cells including neurons and endothelial cells. Therefore, blocking TRPM4 activity during pulmonary injury could alleviate cytokine storm-associated vascular damage.

Cytokines are a diverse group of small proteins that are secreted by cells for intercellular signaling and communication. In a wide variety of infectious and noninfectious diseases, the body's immune system can be overly activated, releasing too many cytokines into the blood in a short time which is termed “cytokine storm”. Multiple organs and systems can be affected by cytokine storm. Among them, acute lung injury is commonly associated with suspected or proven infections in lung and other organs.

A major consequence of cytokine storm is increased vascular permeability. Permeability-inducing factors bind to endothelial cell plasma membrane receptors, and activate heterotrimeric G proteins, causing an increase in intracellular Ca2⁺. This results in myosin-driven endothelial contraction and opening of tight junctions. In lung, excessive extravasation via injured vasculature will lead to pulmonary edema, disrupting gas exchange and causing hypoxia. Patients with severe hypoxia have to rely on ventilators to maintain blood oxygen level. Some of them eventually die of acute respiratory distress syndrome (ARDS) and multiple organ dysfunction.

Cytokine storms are associated with a wide variety of infectious and non-infectious diseases. A number of infectious diseases are generally known to cause cytokine storm, including viral infection associated with cytomegalovirus, Epstein-Barr virus-associated hemophagocytic lympho-histiocytosis, and infectious diseases caused by group A streptococcus, influenza virus, variola virus, and severe acute respiratory syndrome coronavirus (SARS-CoV). Of which, the Coronavirus disease (COVID-19) is an infectious disease caused by the SARS-CoV-2 virus. In COVID-19, cytokine storm is positively correlated with disease severity and mortality. Elderly patients are particularly vulnerable with a much higher chance to develop severe symptoms including pulmonary edema. Patients in the intensive care unit (ICU) usually display increased serum levels of granulocyte colony-stimulating factor, IP-10, MCP-1, macrophage inflammatory protein-1A, and TNF-α, indicating the presence of cytokine storm.

In addition to respiratory pathology, extrapulmonary manifestations of COVID-19 include thrombotic complications, myocardial dysfunction and arrhythmia, acute coronary syndromes, acute kidney injury, gastrointestinal symptoms, hepatocellular injury, hyperglycemia and ketosis, neurologic illnesses, ocular symptoms, and dermatologic complications. These extrapulmonary manifestations and extrapulmonary vascular injuries could lead to stroke, myocardial infarction, and Kawasaki disease. In patients who died from COVID-19-associated respiratory failure, severe endothelial injury was prominent with widespread thrombosis and microangiopathy.

As vascular injury plays a key role during pulmonary injury, there is a need to develop a therapy to protect pulmonary vasculature and prevent or treat pulmonary-related vascular diseases. Vascular protection of the human lungs can reduce pulmonary edema and reduce hypoxia. In addition, vascular protection in other parts of the body other than the lungs, can ameliorate inflammation-associated injury in heart, brain and other organs. When a new virus pandemic, such as COVID-19 arises, it takes time to develop new antiviral drugs and vaccines effective against the pathogen. The outbreak management of a new infectious disease often relies on ameliorating symptoms. Currently, conventional treatment of cytokine storm heavily depends on suppressing the immune system which may generate side effects including the delay of viral pathogen clearance from respiratory tract secretions and inducing secondary infection due to a suppressed immune response. Therefore, there is an urgent need for novel treatments to manage cytokine storm, not only for COVID-19, but also for pandemic caused by other viruses. There is a need to develop new molecules (such as antibodies) and methods to ameliorate vascular damage caused by cytokine storm.

### SUMMARY

In one aspect, the present disclosure refers to a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one aspect, the present disclosure refers to a nucleic acid encoding the antibody or antigen-binding fragment thereof disclosed herein.

In one aspect, the present disclosure refers to an expression vector comprising the nucleic acid disclosed herein.

In one aspect, the present disclosure refers to a host cell comprising the nucleic acid disclosed herein or the expression vector disclosed herein.

In one aspect, the present disclosure refers to a method of producing the antibody or antigen-binding fragment thereof disclosed herein, the method comprising culturing the host cell disclosed herein in a culture medium.

In one aspect, the present disclosure refers to a pharmaceutical composition comprising the antibody or antigen-binding fragment thereof disclosed herein, and a pharmaceutically acceptable carrier, excipient, or diluent.

In one aspect, the present disclosure refers to the antibody or antigen-binding fragment thereof disclosed herein for use as a medicament.

In one aspect, the present disclosure refers to a method of treating cytokine-associated vascular disease, comprising administering to a subject an effective amount of the antibody or antigen-binding fragment thereof disclosed herein, or the pharmaceutical composition disclosed herein.

In one aspect, the present disclosure refers to the use of an effective amount of the antibody or antigen-binding fragment thereof disclosed herein, or the pharmaceutical composition disclosed herein, in the manufacture of a medicament for treating cytokine-associated vascular disease.

In one aspect, the present disclosure refers to a kit for treating cytokine-associated vascular disease comprising the antibody or antigen-binding fragment thereof disclosed herein and/or instructions for use.

### BRIEF DESCRIPTION OF DRAWINGS

The present disclosure will be better understood with reference to the detailed description when considered in conjunction with the non-limiting examples and the accompanying drawings, in which:

FIG. 1 (comprised of FIGS. 1A, 1B and 1C) shows the protein expression of TRPM4 in vascular endothelial cells after inflammation induction. FIG. 1A is a western blot showing the expression of TRPM4 in rat lung microvascular endothelial cells treated with Lipopolysaccharides (LPS) (10 μg/mL), Tumour Necrosis Factor alpha (TNFα) (10 ng/ml) and Interleukin-1β (IL-1β) (10 ng/mL). FIG. 1B is a western blot showing the expression of TRPM4 in human lung microvascular endothelial cells treated with LPS (10 μg/mL), TNFα (10 ng/mL) and IL-1β (10 ng/ml). FIG. 1C is a western blot showing the expression of TRPM4 in human brain microvascular endothelial cells treated with LPS (10 μg/mL), TNFα (10 ng/ml) and IL-1β (10 ng/ml). The western blots are normalized to actin.

FIG. 2 (comprised of FIGS. 2A, 2B, 2C and 2D) are electrophysiology data demonstrating that treatment with endotoxin LPS enhanced hypoxia-induced oncosis in cultured rat lung vascular endothelial cells. FIG. 2A shows that LPS treatment (10 μg/ml) alone increased membrane permeability in cultured rat lung vascular endothelial cell compared to control cells not treated with LPS. FIG. 2B shows that a 7-minute hypoxia treatment increased membrane permeability in cultured rat lung vascular endothelial cell. FIG. 2C shows that the combination of a 7-minute hypoxia and LPS treatment greatly increased membrane permeability in cultured rat lung vascular endothelial cell. FIG. 2D shows that LPS treatment accelerated cell swelling during 7-minute hypoxia induction of cultured rat lung vascular endothelial cell. * p<0.05. Two-way ANAOVA followed by Bonferroni correction; n=8-10 cells.

FIG. 3 (comprised of FIGS. 3A, 3B, 3C and 3D) demonstrates TRPM4 upregulation and activation after LPS treatment-induced oncotic cell death in rat lung vascular endothelial cells.

FIG. 3A shows that a 7-minute hypoxia treatment significantly increased membrane permeability in control IgG and LPS-treated cells. FIG. 3B shows that blocking TRPM4 with a rabbit polyclonal antibody against TRPM4 (M4P) in cells treated with LPS and a 7-minute hypoxia completely abolished the hypoxia and LPS-induced ionic change. FIG. 3C shows that the addition of a rabbit polyclonal antibody against TRPM4 (M4P) significantly inhibited cell volume increase (cell swelling/oncosis) in endothelial cells treated with LPS and a 7-minute hypoxia. FIG. 3D illustrates the percentage of cell death following treatment with 24-hour hypoxia and LPS (at a concentration of 50 or 100 μg/mL) in control cells treated with IgG compared to cells treated with M4P. Cell death was quantified using Trypan blue staining. * p<0.05. *** p<0.001, # p<0.0001. Two-way ANAOVA followed by Bonferroni correction.

FIG. 4 illustrates the effects of a mouse monoclonal antibody (M4M) on human lung vascular endothelial (HLVE) cells after hypoxia and LPS treatment. Calcium imaging of HLVE cells treated with LPS showed reduced current leakage and impeded calcium increase in HLVE cells after hypoxia and LPS treatment. n=56-59 cells.

FIG. 5 (comprised of FIGS. 5A, 5B, 5C, 5D, 5E, 5F, 5G and 5H) shows that blocking TRPM4 with M4P reduced LPS-induced pulmonary inflammation and vascular leakage in mouse lung. FIG. 5A is an example of a hematoxylin and eosin (H&E) staining of a control mouse lung. FIG. 5B is a H&E staining image of a mouse lung 1 day after LPS treatment. FIG. 5C is a western blot showing increased TRPM4 expression in LPS-treated mouse lung compared to control mouse lung, not treated with LPS. FIG. 5D is a H&E staining image of a mouse lung 7 days after LPS and IgG treatment. FIG. 5E is a H&E staining image of a mouse lung 7 days after LPS and M4P treatment. Scale bars: 50 μm. FIG. 5F shows the quantification of Interleukin-6 (IL-6) in bronchoalveolar lavage fluid (BALF) from mouse lung without treatment (Ctrl) or treated with LPS, M4P or IgG. FIG. 5G shows the quantification of IL-1β in BALF from mouse lung without treatment (Ctrl) or treated with LPS, M4P or IgG. As shown in FIGS. 5F and 5G, LPS treatment increased the secretion of IL-6 and IL-1β. FIG. 5H is a survival curve showing the survival percentage of mice receiving intratracheal LPS instillation. Survival of mice after exposure to LPS increased from 33.3% in mice treated with LPS treatment alone compared to 66.7% in mice treated with LPS and M4P.

FIG. 6 (comprised of FIGS. 6A, 6B and 6C) illustrates the characterization of humanized anti-TRPM4 antibodies. FIG. 6A shows the immunofluorescent staining of humanized antibodies in HEK 293 cells transfected with human TRPM4. Human IgG was transfected in HEK 293 as control. Antibody clone A1 is an anti-TRPM4 chimeric antibody. Antibody clones A2-A7 are humanized anti-TRPM4 antibodies. Myc staining showed successful transfection of human TRPM4 and Wheat germ agglutinin (WGA) was used as surface glycoproteins staining. FIG. 6B is a bar graph showing the electrophysiological characterization of the chimeric antibody clone (A1) and the humanized antibody clones (A2-A7). TRPM4 currents at 0 min and 7 min hypoxia were recorded. Currents at +80 mV and −80 mV were summarized in the bar graph. FIG. 6C is a dose-response curve showing dose-dependent inhibition of humanized antibody clone, A2 on hypoxia-induced current increase. IC₅₀: 1.23 μg/ml.

FIG. 7 are sensor-gram plots showing the binding affinity and kinetics of different humanized antibodies to Peptide-biotin. The overlaid smooth black lines represent the kinetic fit of the binding response signals at different antibody concentrations to a 1:1 interaction model.

FIG. 8 (comprised of FIGS. 8A, 8B and 8C) are dynamic light scattering (DLS) thermograms of humanized antibody clones A2 (VH1+VL1), A4 (VH1+VL3) and A6 (VH2+VL1) under different temperatures. FIG. 8A shows that humanized antibody clone A2 (VH1+VL1) was stable at high temperature above 60° C. FIG. 8B shows that humanized antibody clone A4 (VH1+VL3) was stable at high temperature above 60° C. FIG. 8C shows that humanized antibody clone A6 (VH2+VL1) was stable at high temperature above 60° C.

FIG. 9 (comprised of FIGS. 9A, 9B and 9C) are capillary electrophoresis-sodium dodecyl sulfate-non-reduced (CE-SDS-NR) electropherograms of humanized antibody clones A2 (VH1+VL1), A4 (VH1+VL3) and A6 (VH2+VL1) being incubated at 40° C. for 28 days. DO (day 0) indicates the starting point. FIG. 9A shows that humanized antibody clone A2 (VH1+VL1) was stable at 40° C. for 28 days. FIG. 9B shows that humanized antibody clone A4 (VH1+VL3) was stable at 40° C. for 28 days. FIG. 9C shows that humanized antibody clone A6 (VH2+VL1) was stable at 40° C. for 28 days.

FIG. 10 (comprised of FIGS. 10A, 10B and 10C) are CE-SDS-NR electropherograms of humanized antibody clones A2 (VH1+VL1), A4 (VH1+VL3) and A6 (VH2+VL1) which shows the stability of the antibodies under low pH3.5 for 0, 2 and 4 hours. FIG. 10A shows that humanized antibody clone A2 (VH1+VL1) was stable at low pH 3.5 for 4 hours. FIG. 10B shows that humanized antibody clone A4 (VH1+VL3) was stable at low pH 3.5 for 4 hours.

FIG. 10C shows that humanized antibody clone A6 (VH2+VL1) was stable at low pH 3.5 for 4 hours.

### DETAILED DESCRIPTION

The present disclosure describes humanized monoclonal antibodies specific to TRPM4 and their use for treating cytokine-associated vascular diseases. The use of humanized antibodies specific to human TRPM4 is expected to demonstrate enhanced therapeutic effects especially in human patients due to lower risk of immune rejection compared to using rabbit or mouse antibodies known in the art.

In a first aspect, the present disclosure refers to a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one example, the present disclosure refers to the antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO:10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example, each heavy and light chain comprises one variable region and one constant region. It is known in the art that the variable region of the heavy and light chain region of an antibody is for antigen binding. In one example, the variable region comprises three complementarity-determining regions (i.e., CDR1, CDR2 and CDR3) and four framework regions (i.e., FR1, FR2, FR3 and FR4). In one example, the complementarity-determining regions and framework regions are in the order of FR1-CDR1-FR2-CDR2-FR3-CDR3-FR4.

In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 13. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 14. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A2. In one example, an antibody or antigen-binding fragment thereof having a heavy chain variable region comprising an amino acid sequence of SEQ ID NO: 7, and a light chain variable region comprising an amino acid sequence of SEQ ID NO: 11 corresponds to humanized antibody clone A2. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A3. In one example, an antibody or antigen-binding fragment thereof having a heavy chain variable region comprising an amino acid sequence of SEQ ID NO: 7, and a light chain variable region comprising an amino acid sequence of SEQ ID NO: 12 corresponds to humanized antibody clone A3. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13 corresponds to humanized antibody clone A4. In one example, an antibody or antigen-binding fragment thereof having a heavy chain variable region comprising an amino acid sequence of SEQ ID NO: 7, and a light chain variable region comprising an amino acid sequence of SEQ ID NO: 13 corresponds to humanized antibody clone A4. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14 corresponds to humanized antibody clone A5. In one example, an antibody or antigen-binding fragment thereof having a heavy chain variable region comprising an amino acid sequence of SEQ ID NO: 7, and a light chain variable region comprising an amino acid sequence of SEQ ID NO: 14 corresponds to humanized antibody clone A5. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6. In one example, an antibody or antigen-binding fragment thereof having a heavy chain variable region comprising an amino acid sequence of SEQ ID NO: 8, and a light chain variable region comprising an amino acid sequence of SEQ ID NO: 11 corresponds to humanized antibody clone A6. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A7. In one example, an antibody or antigen-binding fragment thereof having a heavy chain variable region comprising an amino acid sequence of SEQ ID NO: 8, and a light chain variable region comprising an amino acid sequence of SEQ ID NO: 12 corresponds to humanized antibody clone A7.

In one example, the present disclosure refers to an antibody or antigen-binding fragment thereof disclosed herein, wherein the antibody or antigen-binding fragment thereof comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO. 55, or SEQ ID NO: 56.

In one example, the present disclosure refers to an antibody or antigen-binding fragment thereof disclosed herein, wherein the antibody or antigen-binding fragment thereof comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 59. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 60. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 58.

In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A2. In one example, an antibody or antigen-binding fragment thereof having a heavy chain comprising an amino acid sequence of SEQ ID NO: 50, and a light chain comprising an amino acid sequence of SEQ ID NO: 57 corresponds to humanized antibody clone A2. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A3. In one example, an antibody or antigen-binding fragment thereof having a heavy chain comprising an amino acid sequence of SEQ ID NO: 50, and a light chain comprising an amino acid sequence of SEQ ID NO: 58 corresponds to humanized antibody clone A3. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59 corresponds to humanized antibody clone A4. In one example, an antibody or antigen-binding fragment thereof having a heavy chain comprising an amino acid sequence of SEQ ID NO: 50, and a light chain comprising an amino acid sequence of SEQ ID NO: 59 corresponds to humanized antibody clone A4. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60 corresponds to humanized antibody clone A5. In one example, an antibody or antigen-binding fragment thereof having a heavy chain comprising an amino acid sequence of SEQ ID NO: 50, and a light chain comprising an amino acid sequence of SEQ ID NO: 60 corresponds to humanized antibody clone A5. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6. In one example, an antibody or antigen-binding fragment thereof having a heavy chain comprising an amino acid sequence of SEQ ID NO: 51, and a light chain comprising an amino acid sequence of SEQ ID NO: 57 corresponds to humanized antibody clone A6. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A7. In one example, an antibody or antigen-binding fragment thereof having a heavy chain comprising an amino acid sequence of SEQ ID NO: 51, and a light chain comprising an amino acid sequence of SEQ ID NO: 58 corresponds to humanized antibody clone A7.

It is known in the art that the constant region of the heavy and light chain region of an antibody comprises a more conserved amino acid sequence compared to the variable region. In one example, the antibody or antigen-binding fragment disclosed herein comprises a constant region selected from the major classes of immunoglobulins, such as IgG, IgD, IgE, IgA and IgM. In one example, the constant region is selected from the group consisting of IgG1, IgG2, IgG3, and IgG4. In one example, the constant region is IgG1. In another example, the constant region is IgG4. In one example, the antibody or antigen-binding fragment thereof disclosed herein comprises a heavy chain comprising an IgG1 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO; 50, SEQ ID NO: 51, SEQ ID NO: 52, SEQ ID NO: 53, SEQ ID NO: 54 or SEQ ID NO: 55. In one example, the antibody or antigen-binding fragment thereof disclosed herein comprises a heavy chain comprising an IgG4 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity SEQ ID NO: 56.

In one example, the present disclosure refers to an antibody or antigen-binding fragment thereof disclosed herein, wherein the antibody specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

In one example, the antibody or antigen-binding fragment thereof disclosed herein inhibits TRPM4 activity. In one example, the antibody or antigen-binding fragment thereof disclosed herein inhibits TRPM4 activity by inhibiting TRPM4 current. In one example, the antibody or antigen-binding fragment thereof disclosed herein inhibits TRPM4 activity by internalizing membrane TRPM4 protein. In one example, the antibody or antigen-binding fragment thereof disclosed herein inhibits TRPM4 activity by inhibiting TRPM4 current and internalizing membrane TRPM4 protein. In one example, under diseased conditions, inhibiting TRPM4 activity refers to blocking, preventing or disrupting the upregulated or activated protein function of TRPM4.

In one aspect, the present disclosure refers to a nucleic acid encoding the antibody or antigen-binding fragment thereof disclosed herein. In one example, the nucleic acid comprises a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOS: 18 and 22, respectively. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 61. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 62. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 63.

In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively corresponds to humanized antibody clone A2. In one example, an antibody or antigen-binding fragment thereof having its heavy chain and its light chain encoded by polynucleotide sequences of SEQ ID NOs: 17 and 21, respectively, corresponds to humanized antibody clone A2. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively corresponds to humanized antibody clone A3. In one example, an antibody or antigen-binding fragment thereof having its heavy chain and its light chain encoded by polynucleotide sequences of SEQ ID NOs: 17 and 22, respectively, corresponds to humanized antibody clone A3. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively corresponds to humanized antibody clone A4. In one example, an antibody or antigen-binding fragment thereof having its heavy chain and its light chain encoded by polynucleotide sequences of SEQ ID NOs: 17 and 23, respectively, corresponds to humanized antibody clone A4. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively corresponds to humanized antibody clone A5. In one example, an antibody or antigen-binding fragment thereof having its heavy chain and its light chain encoded by polynucleotide sequences of SEQ ID NOs: 17 and 24, respectively, corresponds to humanized antibody clone A5. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively corresponds to humanized antibody clone A6. In one example, an antibody or antigen-binding fragment thereof having its heavy chain and its light chain encoded by polynucleotide sequences of SEQ ID NOs: 18 and 21, respectively, corresponds to humanized antibody clone A6. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively corresponds to humanized antibody clone A7. In one example, an antibody or antigen-binding fragment thereof having its heavy chain and its light chain encoded by polynucleotide sequences of SEQ ID NOs: 18 and 22, respectively, corresponds to humanized antibody clone A7.

In one aspect, the present disclosure refers to an expression vector comprising the nucleic acid disclosed herein. Suitable expression vectors for production of recombinant proteins (such as antibodies, such as the humanized antibodies of the present disclosure) are well known to those skilled in the art and examples of expression vectors comprising the nucleic acid disclosed herein include pcDNA3.1, pGEX and pCMV.

In one aspect, the present disclosure refers to a host cell comprising the nucleic acid disclosed herein. In one example, the present disclosure refers to a host cell comprising the expression vector disclosed herein. In one example, the present disclosure refers to a host cell comprising the nucleic acid disclosed herein and the expression vector disclosed herein. In one example, the host cell comprising the nucleic acid disclosed herein and/or the expression vector disclosed herein is human embryonic kidney 293 cells (HEK 293 cells) or Chinese hamster ovary cells (CHO cells). In one example, the host cell comprising the nucleic acid disclosed herein and/or the expression vector disclosed herein may be cells generally used for the production of humanized antibodies which are known to those skilled in the art.

In one aspect, the present disclosure refers to a method of producing the antibody or antigen-binding fragment thereof disclosed herein. In one example, the method comprises culturing the host cell disclosed herein in a culture medium. In one example, the culture medium is Dulbecco's Modified Eagle's Medium (DMEM) (12800017; Thermo Fisher Scientific, USA) supplemented with 10% fetal bovine serum (10500064, Thermo Fisher Scientific, USA); 1.74 g/L sodium bicarbonate; 1.2 g/L HEPES; and 100 U/mL Penicillin-Streptomycin (15140122; Thermo Fisher Scientific, USA), or any other suitable cell culture medium known in the art. In one example, the method further comprises isolating the antibody or antigen-binding fragment thereof from the culture medium. In one example, the method of producing and isolating the antibody or antigen-binding fragment thereof disclosed herein may be methods generally used for the production of recombinant proteins, such as humanized antibodies, which are known to those skilled in the art.

In one example, point mutation(s) has been made to the antibody or antigen-binding fragment thereof disclosed herein to stabilize the resulting antibody clone. In one example, at least one point mutation(s) has been made in a framework region(s) of the antibody to stabilize the resulting antibody clone. In one example, one point mutation has been made in a framework region(s) of the antibody to stabilize the resulting antibody clone. In one example, two point mutations have been made in a framework region(s) of the antibody to stabilize the resulting antibody clone. In one example, three point mutations have been made in a framework region(s) of the antibody to stabilize the resulting antibody clone. In one example, the at least one point mutation(s) can be made in FR1, FR2, FR3 or FR4, or combinations thereof, of the framework region of the antibody. In one example, one point mutation has been made in FR2 of the framework region of the antibody. In one example, two point mutations have been made in FR2 of the framework region of the antibody. In one example, one point mutation has been made in FR3 of the framework region of the antibody. In one example, two point mutations have been made in FR3 of the framework region of the antibody. In one example, three point mutations have been made in FR3 of the framework region of the antibody. In one example, there is no upper limit in the number of mutations made to a framework region of the antibody. In one example, there is no upper limit in the number of mutations made to a framework region of the antibody. In one example, one or more point mutations can be made in any framework region of any heavy chain variable region (such as the heavy chain variable region comprising an amino acid having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10) or any light chain variable region (such as the light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14). In one example, the term “point mutation” refers to a mutation where a single amino acid is substituted, inserted or deleted from an amino acid sequence.

In another example, modification(s) can be made to a constant region of the heavy chain and/or light chain to reduce toxicity. In one example, the modification(s) is point mutation(s). In one example, point mutation(s) has been made to the constant region of the heavy chain of the antibody or antigen-binding fragment thereof disclosed herein to reduce toxicity. In one example, at least one point mutation(s) has been made in the constant region of the heavy chain (or heavy chain constant region) of the antibody to reduce toxicity. In one example, one point mutation has been made in the heavy chain constant region of the antibody to reduce toxicity. In one example, two point mutations have been made in the heavy chain constant region of the antibody to reduce toxicity. In one example, the at least one point mutation(s) can be made in the IgG1, IgG2, IgG3 or IgG4 heavy chain constant region of the antibody or antigen-binding fragment thereof disclosed herein to reduce toxicity. In one example, the at least one point mutation(s) can be made in the IgG1 heavy chain constant region of the antibody or antigen-binding fragment thereof disclosed herein to reduce toxicity. In one example, the at least one point mutation(s) can be made in the IgG4 heavy chain constant region of the antibody or antigen-binding fragment thereof disclosed herein to reduce toxicity. In one example, one point mutation has been made in the IgG1 heavy chain constant region of the antibody to reduce toxicity. In one example, two point mutations have been made in the IgG1 heavy chain constant region of the antibody to reduce toxicity. In one example, one point mutation has been made in the IgG4 heavy chain constant region of the antibody to reduce toxicity. In one example, there is no upper limit in the number of point mutations made to a heavy chain constant region of the antibody. In one example, the LL residues at positions 261 and 262 of IgG1 heavy chain constant region (e.g. positions 261 and 262 of IgG1 heavy chain constant region of VH5) are substituted with AA residues. In one example, substitution of LL residues at positions 261 and 262 of IgG1 heavy chain constant region (e.g. positions 261 and 262 of IgG1 heavy chain constant region of VH5) with AA residues results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54. In one example, the N residue at position 324 of IgG1 heavy chain constant region (e.g. position 324 of IgG1 heavy chain constant region of VH6) is substituted with an A residue. In one example, substitution of N residue at position 324 of IgG1 heavy chain constant region (e.g. position 324 of IgG1 heavy chain constant region of VH6) with an A residue results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55. In one example, the S residue at position 254 of IgG4 heavy chain constant region (e.g. position 254 of IgG4 heavy chain constant region of VH7) is substituted with a P residue. In one example, substitution of S residue at position 254 of IgG4 heavy chain constant region (e.g. position 254 of IgG4 heavy chain constant region of VH7) with a P residue results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56.

In one aspect, the present disclosure refers to a pharmaceutical composition comprising the antibody or antigen-binding fragment thereof disclosed herein, and a pharmaceutically acceptable carrier, excipient, or diluent. In one aspect, the present disclosure refers to the antibody or antigen-binding fragment thereof disclosed herein for use as a medicament.

In one aspect, the present disclosure refers to a method of treating cytokine-associated vascular disease, comprising administering to a subject an effective amount of the antibody or antigen-binding fragment thereof disclosed herein, or the pharmaceutical composition disclosed herein.

In one aspect, the present disclosure refers to use of an effective amount of the antibody or antigen-binding fragment thereof disclosed herein, or the pharmaceutical composition disclosed herein, in the manufacture of a medicament for treating cytokine-associated vascular disease.

In one example, the effective amount of the antibody or antigen-binding fragment thereof is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg. Generally, an effective dosage may be in the range of about 0.1 mg to about 15 mg per kg body weight of the subject; about 0.2 mg to about 14 mg per kg body weight of the subject; about 0.4 mg to about 13 mg per kg body weight of the subject; about 0.6 mg to about 12 mg per kg body weight of the subject; about 0.8 mg to about 11 mg per kg body weight of the subject; or about 1 mg to about 10 mg per kg body weight of the subject; or about 2 mg to about 9 mg per kg body weight of the subject; about 3 mg to about 8 mg per kg body weight of the subject; about 4 mg to about 7 mg per kg body weight of the subject; or about 0.1 mg per kg body weight of the subject, or about 0.2 mg per kg body weight of the subject; or about 0.4 mg per kg body weight of the subject, or about 0.6 mg per kg body weight of the subject, or about 0.8 mg per kg body weight of the subject, or about 1 mg per kg body weight of the subject, or about 2 mg per kg body weight of the subject, or about 3 mg per kg body weight of the subject, or about 4 mg per kg body weight of the subject, or about 5 mg per kg body weight of the subject, or about 6 mg per kg body weight of the subject, or about 7 mg per kg body weight of the subject, or about 8 mg per kg body weight of the subject, or about 9 mg per kg body weight of the subject, or about 10 mg per kg body weight of the subject, or about 11 mg per kg body weight of the subject, or about 12 mg per kg body weight of the subject, or about 13 mg per kg body weight of the subject, or about 14 mg per kg body weight of the subject, or about 15 mg per kg body weight of the subject.

In one example, the concentration of the antibody or antigen-binding fragment thereof required to inhibit TRPM4 activity in vitro is from 1 μg/ml to 5 μg/ml, or from 2 μg/ml to 4 μg/ml, or about 1 μg/ml, or about 2 μg/ml, or about 3 μg/ml, or about 4 μg/ml, or about 5 μg/ml. In one example, the concentration of the antibody or antigen-binding fragment thereof required to inhibit TRPM4 activity in vitro is 1.23 μg/ml. In one example, the concentration of the antibody or antigen-binding fragment thereof required to inhibit TRPM4 activity in vitro may be determined by a dose-dependent assay detecting for the dose-dependent effect of the antibody or antigen-binding fragment thereof on hypoxia-induced TRPM4 current increase.

In one example, the effective amount of the antibody or antigen-binding fragment thereof treats cytokine-associated vascular disease by preventing vascular damage in the subject. In one example, the vascular damage is prevented by preventing hypoxia and inflammation-induced cell death of vascular cells. In one example, the vascular cells are pulmonary endothelial cells. In one example, the vascular cells are cerebral vascular endothelial cells. In one example, the vascular cells are pulmonary and cerebral vascular endothelial cells.

In one example, the cytokine-associated vascular disease is selected from the group consisting of pulmonary edema, extrapulmonary vascular injury, pulmonary inflammation, lung infection, viral infection associated with pulmonary edema, bacterial infection associated with pulmonary edema, and fungal infection associated with pulmonary edema. In one example, the cytokine-associated vascular disease is pulmonary inflammation.

In one example, the viral infection associated with pulmonary edema is associated to a virus selected from the group consisting of cytomegalovirus, Epstein-Barr virus, influenza virus, variola virus and severe acute respiratory syndrome coronavirus (SARS-CoV). In one example, the virus is SARS-CoV. In one example, the viral infection associated with pulmonary edema is associated to viruses that cause pulmonary injury.

In one example, the bacterial infection associated with pulmonary edema is associated to a bacteria selected from the group consisting of group A Streptococcus, Haemophilus bacteria, Staphylococcus aureus and Mycobacterium tuberculosis.

In one example, the fungal infection associated with pulmonary edema is associated to a fungus selected from the group consisting of Aspergillus, Cryptococcus, Pneumocystis and endemic fungi. In one example, the endemic fungus is selected from the group consisting of Sporothrix schenckii, Coccidioides immitis and Coccidioides posadasii, Paracoccidioides, Blastomyces dermatitidis and Histoplasma capsulatum. In one example, endemic fungi are distributed in the environment of certain geographical areas. Infection of humans by endemic fungi is mainly caused by inhalation or by damage of the skin. In one example, endemic fungal disease may be found in both immunocompromised subjects and normal subjects with a spectrum of symptoms.

In one aspect, the present disclosure refers to a kit for treating cytokine-associated vascular disease comprising the antibody or antigen-binding fragment thereof disclosed herein and/or instructions for use. In some examples, the reagents provided in the kit as described herein may be provided in separate containers comprising the components independently distributed in one or more containers.

As used in this application, the singular form “a,” “an,” and “the” include plural references unless the context clearly dictates otherwise.

As used herein, the term “at least” when used in the context of a value is understood to mean “greater than or equal to” said value. For example, a sequence having “at least 85% sequence identity” to another sequence would be understood to include a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the other sequence. In another example, an antibody or antigen-binding fragment thereof comprising a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 would be understood to include an antibody or antigen-binding fragment thereof comprising a heavy chain variable region of SEQ ID NO: 7 (i.e. 100% sequence identity), and a light chain variable region of SEQ ID NO: 11 (i.e. 100% sequence identity).

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

The term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies of the present disclosure may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F(ab)₂, scFv (single chain Fv), dAb (single domain antibody), bi-specific antibodies, diabodies and triabodies As used in this application, “antibody fragment” refers to an antibody which has been reduced in length by one or more amino acids while retaining its antigenic activity.

The term “humanized antibody” refers to antibodies from non-human species whose protein sequences have been modified to increase their similarity to antibody variants produced naturally in humans.

The term “antibody variant” as used herein refers to antibodies with differences in their amino acid sequences but still retain their binding specificities to their antigens. For example, one or more conservative amino acid substitutions may be made to the TRPM4 antibody or its antigen-binding fragment thereof while retaining binding specificity to TRPM4.

As used herein “sequence identity” refers to the residues in two sequences that are the same when aligned for maximum correspondence over a specified window of comparison by means of computer programs known in the art such as GAP provided in the GCG program package (Program Manual for the Wisconsin Package, Version 11, 2005, Genetics Computer Group, 575 Science Drive, Madison, Wisconsin, USA 53711) and DNAstar Lasergene.

As used herein the term “treatment” refers to any and all uses which remedy a disease state or symptoms, prevent the establishment of disease, or otherwise prevent, hinder, retard, or reverse the progression of disease or other undesirable symptoms in any way whatsoever.

In the context of this specification, the term “an effective amount” includes within its meaning a non-toxic but sufficient amount of an agent (for example, the antibody or its antigen-binding fragment thereof of the present disclosure) to provide the desired effect. The exact amount required will vary from subject to subject depending on factors such as the species being treated, the age and general condition of the subject, the particular agent (for example, the antibody or its antigen-binding fragment thereof of the present disclosure) being administered and the mode of administration and so forth. Thus, it is not possible to specify an exact “effective amount”. However, for any given case, an appropriate “effective amount” may be determined by one of skill in the art using appropriate means.

In one example, the antibody or its antigen-binding fragment thereof may be administered to a subject in need thereof. Convenient modes of administration include injection (subcutaneous, intravenous, etc.), oral administration, inhalation, transdermal application, topical creams or gels or powders, or rectal administration. Depending on the route of administration, the antibody or its antigen-binding fragment thereof may be coated with a material to protect it from the action of enzymes, acids and other natural conditions which may inactivate its therapeutic activity. The antibody or its antigen-binding fragment thereof may also be administered parenterally or intraperitoneally.

Dispersions of the antibody or its antigen-binding fragment thereof according to the present disclosure may also be prepared in glycerol, liquid polyethylene glycols, and mixtures thereof and in oils. Under ordinary conditions of storage and use, pharmaceutical preparations may contain a preservative to prevent the growth of microorganisms.

The term “pharmaceutical composition” refers to a preparation containing a pharmaceutically active ingredient which is suitable for administration to a subject. The term “pharmaceutically acceptable carrier” refers to an ingredient in a pharmaceutical composition, other than the pharmaceutically active ingredient, which is nontoxic to a subject. The use of such “pharmaceutically acceptable carrier” is well known in the art. A pharmaceutically acceptable carrier includes, but is not limited to, a buffer, excipient, stabilizer, or preservative. The antibody or its antigen-binding fragment thereof may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the antibody or its antigen-binding fragment thereof may be administered by injection. In the case of injectable solutions, the carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, isotonic agents, for example, sugars, polyalcohols such as mannitol, sorbitol, sodium chloride may be included in the pharmaceutical composition. Prolonged absorption of the injectable pharmaceutical compositions can be brought about by including in the pharmaceutical composition an agent which delays absorption, for example, aluminium monostearate and gelatin.

Sterile injectable solutions can be prepared by incorporating the analogue in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Single or multiple administrations of the pharmaceutical compositions according to the present disclosure may be carried out. One skilled in the art would be able, by appropriate means, to determine effective, non-toxic dosage levels of the antibody or its antigen-binding fragment thereof and/or pharmaceutical composition of the present disclosure and an administration pattern which would be suitable for treating the diseases and/or infections to which the antibody or its antigen-binding fragment thereof and pharmaceutical compositions are applicable.

Further, it will be apparent to one of ordinary skill in the art that the optimal course of treatment, such as the number of doses of the antibody or its antigen-binding fragment thereof or pharmaceutical composition of the present disclosure given per day for a defined number of days, can be ascertained using convention course of treatment determination tests.

As used herein, the term “increase” refers to a rise in amount, expression level or number on a positive scale. Conversely, the term “decrease” indicates a change on a negative scale.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the present disclosure embodied therein herein disclosed may be resorted to by those skilled in the art, and that such modifications and variations are considered to be within the scope of this present disclosure.

The disclosure has been described broadly and generically herein. Each of the narrower species and subgeneric groupings falling within the generic disclosure also form part of the present disclosure. This includes the generic description of the present disclosure with a proviso or negative limitation removing any subject matter from the genus, regardless of whether or not the excised material is specifically recited herein.

Other embodiments are within the following claims and non-limiting examples.

### EXAMPLES

### Methods

### Antibody Humanization by CDR Grafting Plus Back Mutation

The structure of parental antibody was modelled by computer-aided homology modelling program. Humanized antibodies were designed using CDR grafting. Briefly, the CDRs of parental antibody were grafted into the human acceptors to obtain humanized light chains and humanized heavy chains for each parental antibody. 4 heavy chains (VH1, VH2, VH3 and VH4) and 4 light chains (VL1, VL2, VL3 and VL4) were paired with each other for affinity ranking experiment.

### Production of Chimeric and Humanized Antibodies

The DNA sequences encoding the chimeric and humanized antibodies heavy and light chains were synthesized and inserted into pcDNA3.4 vector to construct expression plasmids of full-length IgGs. The designed plasmids of heavy and light chain were sent for transfection to HEK 293 or CHO cells using Lipofectamine 2000. The culture media for both HEK 293 and CHO cells: Dulbecco's Modified Eagle's Medium (DMEM) (12800017; Thermo Fisher Scientific, USA) supplemented with 10% fetal bovine serum (10500064, Thermo scientific); 1.74 g/L sodium bicarbonate; 1.2 g/L HEPES; and 100 U/mL Penicilin-Streptomycin (15140122; thermo scientific). The recombinant IgGs secreted to the medium were purified using protein. The purified antibody was buffer-exchanged into PBS using PD-10 desalting column. The concentration and purity of the purified protein were determined by OD280 and SDS-PAGE, respectively.

### Affinity Ranking of Chimeric and Humanized Antibodies

For affinity ranking, antibodies were captured on the sensor chip through Fc capture method. Peptide-biotin was used as the analyte. The surface was regenerated before the injection of another antibody. The process was repeated until all antibodies are analyzed. The off-rates of antibodies were obtained from fitting the experimental data locally to 1:1 interaction model using the Biacore 8K evaluation software. The antibodies were ranked by their dissociation rate constants (off-rates, kd). Based on the ranking result, the top 3 clones were selected.

### Affinity Measurement of Purified Humanized IgGs

The affinity of purified antibody binding to Peptide-biotin was individually determined using a Surface Plasmon Resonance (SPR) biosensor, Biacore 8K (GE Healthcare). Antibodies were captured on the sensor chip through Fc capture method. Peptide-biotin was used as the analyte. The data of dissociation (kd) and association (ka) rate constants were obtained using Biacore 8K evaluation software. The equilibrium dissociation constants (KD) were calculated from the ratio of kd over ka.

### Western Blot for Lung Tissue

Whole mice lung was homogenized and lysed in 200 μL 1% Triton-X 100 (1610407, Bio-Rad, CA, USA) and Protease Inhibitor Cocktail Tablets (04693124001, Roche, Switzerland) in Phosphate-buffered Saline (NaCl, KCl, Na₂HPO₄, KH₂PO₄) in Eppendorf tubes. The tubes were rotated at 20 rpm for 15 minutes at 4° C. and then centrifuged at 13,500 rpm for 15 minutes at 4° C. The pellets were lysed in 200 μL 2% Sodium dodecyl sulfate, SDS (L5750, Sigma-Aldrich, MI, USA) and Protease Inhibitor Cocktail Tablets (04693124001, Roche, EU) in PBS at room temperature. The pellets were then sonicated using Ultrasonic Processor (VCX130, Sonics, USA) at 40 Watt until pellet can no longer be seen. The tubes were centrifuged at 13,500 rpm for 5 minutes at room temperature. The supernatant (insoluble protein fraction) was kept and to be stored at room temperature for western blotting. 100 μg protein was resolved on 10% SDS-PAGE gels at 80 V, and electrophoretically transferred to PVDF membranes (1620177, Bio-Rad, CA, USA) at 100 V for 2 hours at 4° C. After blocking with StartingBlock (PBS) blocking buffer (37538, Thermo Fisher Scientific, MA, USA) for 1 hour at room temperature, the membranes were incubated overnight at 4° C. with primary antibodies: TRPM4 (ACC-044, Alomone, 1:300) and anti-actin (A1978, Sigma-Aldrich, MI, USA, 1:1000). After washing away primary antibodies, the membranes were incubated with secondary antibodies against mouse or rabbit IgG for 1 hour at room temperature. Primary and secondary antibodies were prepared in StartingBlock (PBS) blocking buffer with 0.05% Tween®20 (P7949, Sigma-Aldrich, MI, USA). Washing buffers contained 0.1% Tween®20 dissolved in PBS. Amersham ECL Western Blotting Analysis System (RPN2109, GE Healthcare, IL, USA) was used and the bands were visualized using a medical X-ray processor (MXP-2000, KODAK, NY, USA).

### Western Blot for Lung Microvascular Endothelial Cells

Rat lung microvascular endothelial cells (RLMEC) cells grown in 60 mm petri dishes were treated with 10 μg/mL LPS (Lipopolysaccharides from Escherichia coli 0111: B4) (L4391, Sigma-Aldrich, MI, USA), 10 ng/mL TNF-alpha (Tumor Necrosis Factor-Alpha Human) (T6674, Sigma-Aldrich, MI, USA) and 10 ng/mL IL-1beta (Interleukin 1 beta human) (SRP3083, Sigma-Aldrich, MI, USA). 48 hours after treatment, 80 μg of total protein was resolved on 10% SDS-PAGE gels at 80 V, and electrophoretically transferred to PVDF membranes (1620177, Bio-Rad, CA, USA) at 100 V for 2 hours at 4° C. After blocking with StartingBlock (PBS) blocking buffer (37538, Thermo Fisher Scientific, MA, USA) for 1 hour at room temperature, the membranes were incubated overnight at 4° C. with primary antibodies: TRPM4 (ACC-044, Alomone, 1:300) and anti-actin (A1978, Sigma-Aldrich, MI, USA, 1:1000). After washing away primary antibodies, the membranes were incubated with secondary antibodies against mouse or rabbit IgG for 1 hour at room temperature. Primary and secondary antibodies were prepared in StartingBlock (PBS) blocking buffer with 0.05% Tween20 (P7949, Sigma-Aldrich, MI, USA). Washing buffers contained 0.1% Tween20 dissolved in phosphate-buffered saline (PBS). Amersham ECL Western Blotting Analysis System (RPN2109, GE Healthcare, IL, USA) was used and the bands were visualized using a medical X-ray processor (MXP-2000, Kodak, NY, USA). Quantification was done using ImageJ.

### Immunostaining and Hematoxylin and Eosin (H&E) Staining

To evaluate the histological alterations, lung tissues were fixed with 10% buffered formalin for 24 hours, embedded in paraffin, and sectioned at 4-μm thickness. After deparaffinization and dehydration, the sections were stained with haematoxylin and eosin (H&E) using standard histological techniques. Briefly, after deparaffinizing and rehydrating the slides in distilled water, the slides were stained in Mayers Hematoxylin for 1 minute. The slides were subsequently washed 4 to 5 times until blue stain stopped coming off the slides and incubated in 1×PBS for 1 minute. The slides were then washed 3 times with distilled water and counterstained in Alcoholic-Eosin for 1 minute. After washing, the slides were dehydrated through 3 changes of 95% EtOH and 2 changes of 100% EtOH for 1 minute each and incubated 3 times in Xylene for 1 minute each. The slides were then mounted on coverslips for subsequent analysis. Morphological changes in lung tissue were observed under a light microscope to evaluate the degree of lung injury.

### Hematoxylin & Eosin (H&E) Staining

The normoxic and hypoxic cortical neurons grown on coverslips were incubated with Neurobasal (NB) medium containing 20.8 μg/mL of the TRPM4 antibody or control IgG for 40 minutes at 37° C. The cells were fixed with 4% (v/v) paraformaldehyde (PFA) for 20 minutes at room temperature followed by permeabilization with 0.1% (v/v) Triton X-100 for 15 minutes. The cells were then incubated 5% fetal bovine serum (FBS) for 30 minutes and then incubated with the appropriate Alexa Fluor® 594 secondary antibody prepared in 5% FBS/PBS for 1 hour at room temperature to detect TRPM4 or control IgG. The cells were incubated with mouse anti-MAP2 antibody (M9942; Sigma-Aldrich) or rabbit anti-MAP2 (M3696; Sigma-Aldrich) prepared at 1:800 dilution in 5% FBS/PBS for 1 hour. After which, the cells were added with the corresponding Alexa Fluor® 488 secondary antibody to anti-MAP2 prepared in 5% FBS/PBS for 1 hour. The cells were counterstained with DAPI for 5 mins. The cells were washed with PBS between incubations and before mounting on glass slides using the FluorSave™ reagent (Merck, NJ, USA). Fluorescent images were captured with a laser scanning confocal microscope system.

### Electrophysiology in Cells

Whole-cell patch clamp was used to measure currents in cells grown in 24-well plates. Patch electrodes were pulled using a Flaming/Brown micropipette puller (P-1000, Sutter Instrument, CA, USA) and polished with a microforge (MF-200, WPI Inc. FL, USA). Whole-cell currents were recorded using a patch clamp amplifier (Multiclamp 700B equipped with Digidata 1440A, Molecular Devices, CA, USA). The aCSF bath solution contained (in millimole/liter): NaCl 140, CaCl₂)₂, KCl₂, MgCl₂ 1, glucose 20 and HEPES 20 at pH 7.4. The internal solution contained (in millimole/liter): CsCl 156, MgCl₂ 1, EGTA 10 and HEPES 10 at pH 7.2 adjusted with CsOH.

Cells were treated with 10 mg/ml LPS w/o 20.8 mg/ml IgG, M4M (for human lung microvascular endothelial cells (HLMEC)) or M4P (for RLMEC) for 24 hours. Ischemia/Hypoxia was induced by applying a bath solution containing 5 mM NaN₃ and 10 mM 2-deoxyglucose (2-DG) continuously through a MicroFil (34 Gauge, WPI Inc. USA) around 10 μm away from the recording cells. The flow rate was 200 μl/minute. The current-voltage relations were measured by applying voltage for 200 ms from −80 to +80 mV at a holding potential of 0 mV. The sampling rate was 20 kHz and the filter setting was 1 KHz. Data were analyzed using pClamp10, version10.2 (Molecular Devices, CA, USA).

### Calcium Imaging

The calcium ion influx in the cells was measured using a Ca²⁺ binding dye, Fura-2-acetoxymethyl ester (Fura-2AM) (F1201; Thermo Fisher). Cells were loaded with 1 mM Fura-2AM added to the culture medium for 30 minutes in the dark in a cell culture humidified incubator set at 37° C. with 5% CO₂. Cells were then gently washed with the physiological buffer solution, pH7.3 (140 mM NaCl, 5 mM KCl, 1.8 mM CaCl₂·2H₂O, 1 mM MgCl₂·6H₂O, 10 mM HEPES, 10 mM D-Glucose) to remove the Fura-2 AM. Then, the cells were placed on the Olympus IX81 microscope with the MT-20 illumination unit, and the cells were continuously superfused with the physiological buffer solution until 20 mg/ml LPS prepared in the physiological buffer solution, followed by a continuous flow of the physiological buffer solution. Fura-2 ratio images were taken at alternating excitation wavelengths of 350 nm and 380 nm every 0.4 seconds and emission at 510 nm. Intracellular calcium concentration was calculated from the ratio of Fura-2 dye wavelengths using the Olympus Xcellence RT software.

For experiments conducted in normoxia, cells were treated with 10 mg/ml IgG, M4M (for HLMEC) or M4P (for RLMEC) for 24 hours. For experiments conducted in hypoxia, cells were treated with LPS (10 μg/ml) w/o IgG or M4M (M4P) for 24 hours in hypoxia induction chamber.

### Dynamic Light Scattering (DLS)

Antibodies were dissolved using a phosphate buffered saline to make a concentration of 3 mg/ml. The samples were then loaded into capillaries. Thermal ramps were run from 25° C. to 70° C. in 1° C. increment. Measurement was performed using a DLS system (Wyatt, DynaPro Plate Reader III).

### Non-Reducing Capillary Electrophoresis-Sodium Dodecyl Sulfate (CE-SDS-NR)

Antibodies were dissolved using a phosphate buffered saline to make a concentration of 3 mg/ml. For high temperature test, the samples were incubated at 40° C. for 7, 14, and 28 days. For low pH stability test, the solution pH was adjusted to pH3.5, and the samples were incubated for 2 and 4 hours at 37° C. After incubation, 100 μg of all samples was obtained to prepare the loading sample solution by adding the sample buffer (phosphate salt, citric acid and SDS), followed by the addition of 10 kD Internal Standard marker and alkylating reagent-NEM (N-Ethylmaleimide). The samples were then incubated at 70° C. for 10 min and allowed to cool down to room temperature. The resulting solutions were centrifuged, and the supernatants were taken for analysis on the CE system (SCIEX, P/ACE MDQ plus).

### Results

Analysis of TRPM4 Expression in Vascular Endothelial Cells after Inflammation Induction

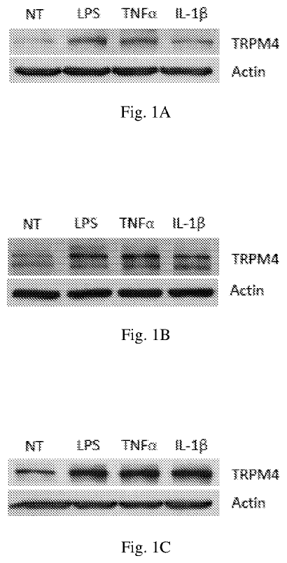

To examine the expression of TRPM4 channel under inflammation conditions and during cytokine storm, vascular endothelial cells from different species were treated with LPS (10 μg/mL), TNFα (10 ng/ml) or IL-1β (10 ng/mL), which are commonly used for inducing pulmonary inflammation. These vascular endothelial cells include rat lung microvascular endothelial cells, human lung microvascular endothelial cells, and human brain microvascular endothelial cells. In these cells, treatment with LPS, TNFα and IL-1β induced TRPM4 upregulation (FIGS. 1A, 1B and 1C), indicating that inflammation could stimulate TRPM4 expression in both pulmonary and extrapulmonary vasculature.

### Effects of LPS on Oncotic Cell Death of Rat Lung Vascular Endothelial Cells

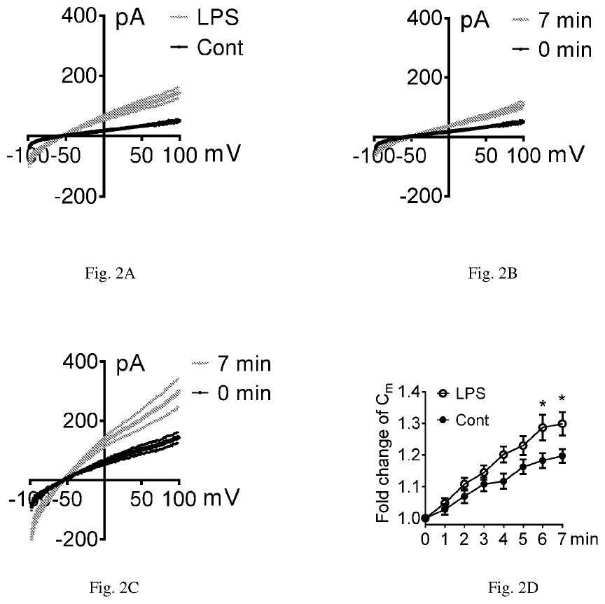

As TRPM4 can be activated by hypoxic condition and pneumonia is known to reduce oxygen exchange, it is possible that TRPM4 could participate in the cell death in vascular cells during pneumonia. LPS is an endotoxin found in the outer membrane of Gram-negative bacteria, and is widely used to induce cytokine storm. To examine the effect of LPS on oncotic cell death, cultured rat pulmonary vascular endothelial cells were treated with LPS. LPS treatment (10 μg/ml) alone increased membrane permeability (FIG. 2A). A 7-minute hypoxia treatment also increased membrane permeability in control endothelial cells (FIG. 2B). A combination of 7-minute hypoxia and LPS treatment greatly increased membrane permeability in cultured endothelial cells (FIG. 2C). LPS treatment accelerated cell swelling in cultured endothelial cells during a 7-minute hypoxia treatment (FIG. 2D). Collectively, the results shows that LPS treatment enhanced hypoxia-induced oncosis in cultured rat lung vascular endothelial cells.

### Analysis of TRPM4 Expression and Activity Under Hypoxia and Inflammation Conditions

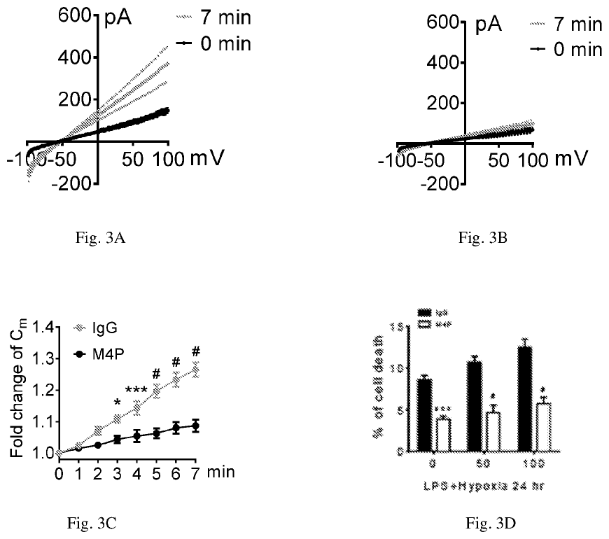

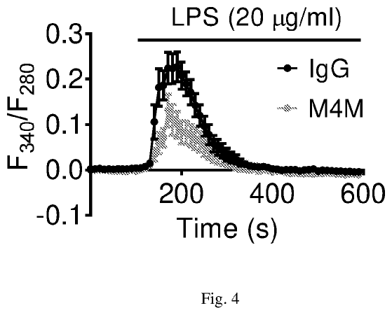

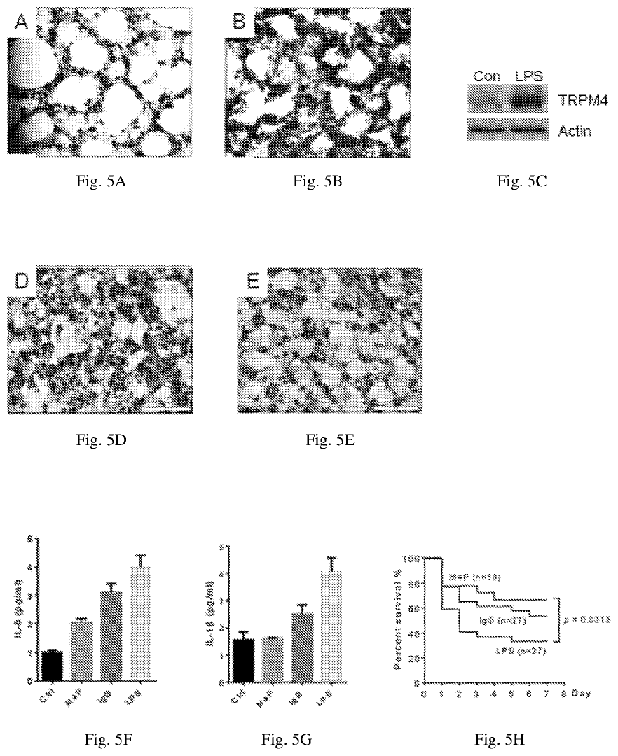

To determine the effects of TRPM4 expression and activity under hypoxia and inflammation conditions, rat pulmonary vascular endothelial cells were exposed to different conditions to induce hypoxia and/or inflammation. In rat pulmonary vascular endothelial cell treated with control IgG and LPS, a 7-minute hypoxia treatment significantly increased ionic permeability (FIG. 3A). In rat pulmonary vascular endothelial cell treated with M4P (to inhibit TRPM4 activity) completely abolished the hypoxia and LPS-induced ionic change (FIG. 3B). In the presence of both hypoxia (7-minute hypoxia treatment) and LPS, cell swelling (oncosis) was observed to develop gradually, and the addition of M4P significantly inhibited cell volume increase (cell swelling/oncosis) (FIG. 3C). Cell death was quantified using Trypan blue staining following treatment with 24-hour hypoxia and LPS (at a concentration of 50 or 100 μg/mL) (FIG. 3D). Collectively, the results indicates that blocking TRPM4 with M4P significantly reduced cell death at all concentrations of LPS.

### Effects of TRPM4 Specific Mouse Antibody on Human Lung Vascular Endothelial (HLVE) Cells Exposed to Hypoxia and LPS Treatment

It is known that human and rodent TRPM4 channels share around 56% of homology. Calcium imaging of HLVE cells showed that the addition of M4M (specific to rodent TRPM4) to HLVE cells reduced current leakage and inhibited calcium increase in HLVE cells after a 7-minute hypoxia and LPS treatment (FIG. 4). This indicates that TRPM4 has cross-species role in pulmonary vascular damage during hypoxia and inflammation.

### TRPM4-Blocking Antibody Reduced Pulmonary Inflammation and Vascular Leakage in Mouse Lung

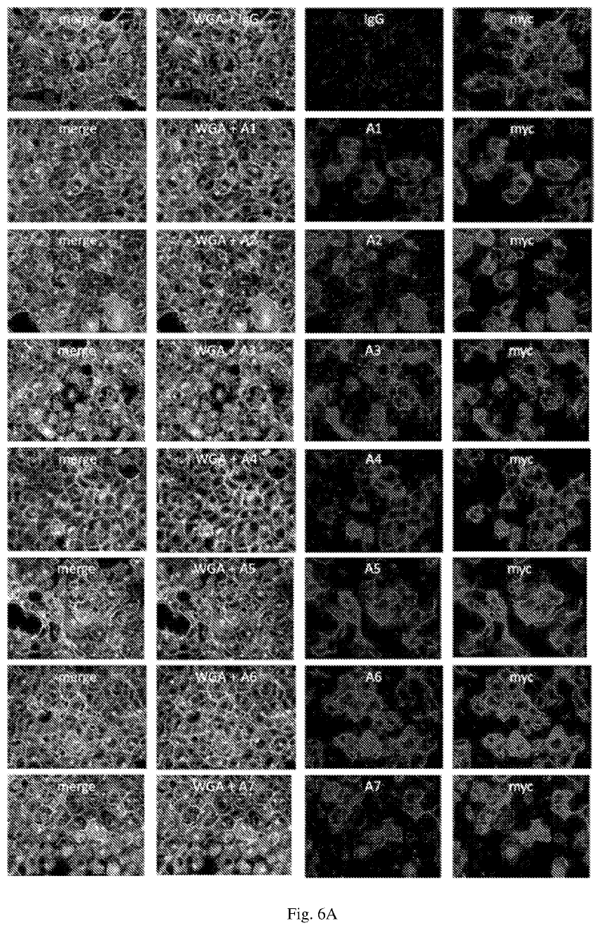

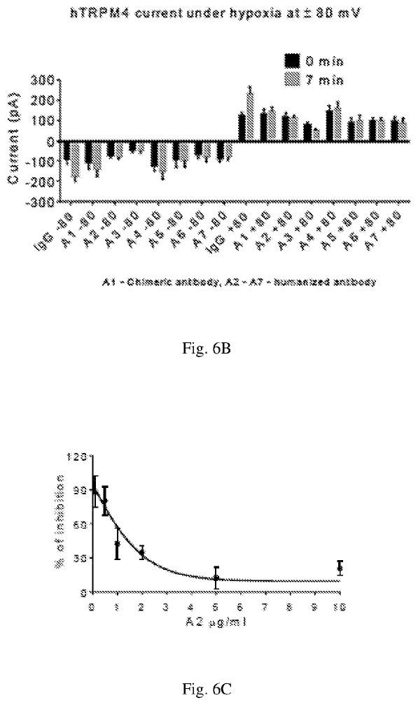

In comparison with untreated control mouse lung (FIG. 5A), LPS instillation for 1 day induced significant interstitial hemorrhage, infiltration of inflammatory cells, and a thickened alveolar wall in mouse lung sample (FIG. 5B). Western Blotting analysis shows that LPS treatment increased TRPM4 expression in the lungs of mice (FIG. 5C). Further, 7 days after LPS instillation, thickened alveolar wall was still prominent in control IgG-treated mouse lung sample (FIG. 5D). On the other hand, blocking TRPM4 with M4P significantly ameliorated pulmonary inflammation (FIG. 6E). Inflammatory responses were quantified in bronchoalveolar lavage fluid (BALF). As shown in FIG. 5F and FIG. 5G, LPS increased the secretion of IL-6 and IL-1β and blocking TRPM4 significantly reduced the expression of IL-6 and IL-1β. Survival of mice after exposure to LPS was increased from 33.3% in mice treated with LPS treatment alone compared to 66.7% in mice treated with LPS and M4P (FIG. 5H).

### Validation of Antibody Binding Affinity of Humanized Antibodies Against TRPM4

Humanized antibodies were generated by replacing the framework regions (FRs) from mouse M4M antibody with counterparts from human, while maintaining the antibody's complementarity-determining regions (CDRs) binding regions. The antibody binding affinity of the chimeric and humanized antibodies was assessed by surface plasmon resonance (SPR) as shown in Table 1. Antibody clone A1 is the chimeric antibody and antibody clones A2-7 are humanized antibodies. Based on the results obtained from SPR, humanized clone A2 has the strongest SPR signal. The antigen binding kinetics to the chimeric and humanized antibodies are shown in Table 2. For affinity ranking, antibodies were captured on the sensor chip through Fc capture method. Peptide-biotin was used as the analyte. The surface was regenerated before the injection of another antibody. The process was repeated until all antibodies are analyzed. The off-rates of antibodies were obtained from fitting the experimental data locally to 1:1 interaction model using the Biacore 8K evaluation software. The antibodies were ranked by their dissociation rate constants (off-rates, kd). Based on the ranking result, the top humanized clones were selected. Table 3 shows the humanization design and back mutation sites in the framework regions of the heavy and light chain regions of the humanized antibody clones. Bolded and underlined amino acids correspond to the back mutation sites in the framework regions.

Humanized antibody clones (A2-A7) were generated by combining various heavy and light chain variable domains (VH1-VH4 variable regions and VL1-VL4 variable regions). The amino acid sequence of the variable domains for heavy and light chains of the humanized antibodies are as follows:

```
VH1 variable region:
  (SEQ ID NO: 7)
QVQLQESGPGLVKPSQTLSLTCTVSGYSITSDYAWNWIRQHPGKGLEWI
GYLSYSGVTSYNPSLKGRVTISVDTSKNQFSLKLSSVTAADTAVYYCAR
KGTYYRYEGSYWYFD
VWGQGTTVTVSS
VH2 variable region:
  (SEQ ID NO: 8)
QVQLQESGPGLVKPSQTLSLTCTVSGYSITSDYAWNWIRQHPGKGLEWM
GYLSYSGVTSYNPSLKGRVTISRDTSKNQFSLKLSSVTAADTAVYYCAR
KGTYYRYEGSYWYFDVWGQGTTVTVSS
VL1 variable region:
(SEQ ID NO: 11)
DIQMTQSPSSLSASVGDRVTITCHANQNIDVWLSWYQQKPGKAPKLLIY
KTSNLHTGVPSRFSGSGSGTDFTFTISSLQPEDIATYYCQQGQSYPLTF
GGGTKLEIK
VL2 variable region:
(SEQ ID NO: 12)
DIQMTQSPSSLSASVGDRVTITCHANQNIDVWLSWYQQKPGKAPKLLIY
KTSNLHTGVPSRFSGSGSGTDFTLTISSLQPEDIATYYCQQGQSYPLTF
GGGTKLEIK
VL3 variable region:
(SEQ ID NO: 13)
DIQMTQSPSSLSASVGDRVTITCHANQNIDVWLSWYQQKPGNAPKLLIY
KTSNLHTGVPSRFSGSGSGTDFTFTISSLQPEDIATYYCQQGQSYPLTF
GGGTKLEIK
VL4 variable region:
(SEQ ID NO: 14)
DIQMTQSPSSLSASVGDRVTITCHANQNIDVWLSWYQQKPGNAPKLLIY
KTSNLHTGVPSRFSGSGSGTDFTLTISSLQPEDIATYYCQQGQSYPLTF
GGGTKLEIK
```

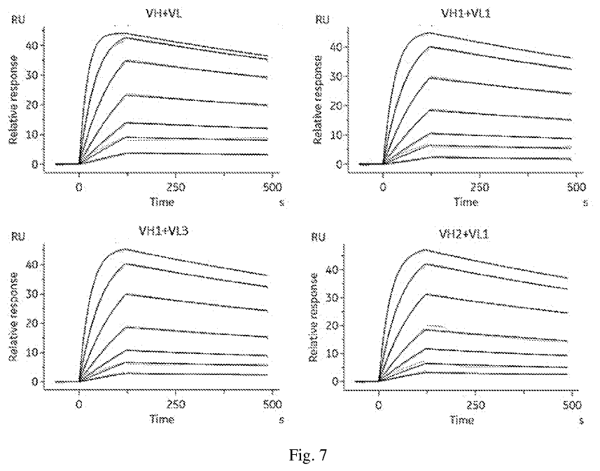

Biochemical and functional characterization were performed to evaluate the antigenic properties of the humanized antibodies. Immunofluorescent staining showed that all six humanized antibodies A2-7 and the chimeric antibody A1 could stain human TRPM4 in transfected HEK 293 cells (FIG. 6A). In addition to staining cytosolic TRPM4, colocalization with surface marker WGA suggested that these antibodies also recognized surface TRPM4. Next, functions of the humanized antibodies were evaluated by electrophysiology (FIGS. 6B and 6C). Human TRPM4 current was significantly elevated by a 7-minute hypoxia treatment in control IgG treated cells (FIG. 6B). All 6 humanized antibodies (A2-A7) and the chimeric antibody A1 successfully inhibited hypoxia-induced current increase at a concentration of 20 μg/ml. Since humanized antibody A2 has the least number of back mutation sites while maintaining excellent binding affinity, the dose-dependent effect of A2 on hypoxia-induced TRPM4 current increase was further determined (FIG. 6C). Using human IgG as a control, the IC₅₀ of A2 was determined to be 1.23 μg/ml in a dose-dependent inhibition assay. No difference was be found when the dose was increased above 5 μg/ml (FIG. 6C). In comparison with the dose of 20.8 μg/ml that was required for M4M or M4P to inhibit hypoxia-induced current increase, the results indicated that humanized antibody clone A2 has better sensitivity in blocking TRPM4 compared to its mouse or rabbit antibody counterpart. The binding affinity and kinetics of different humanized antibodies (A1, A2, A4 and A6) to Peptide-biotin were also shown in the form of sensor-grams (FIG. 7). The overlaid smooth black lines of the sensor-grams represent the kinetic fit of the binding response signals at different antibody concentrations to a 1:1 interaction model.

TABLE 1
Binding kinetics of antibody to antigen

| Ligand | Analyte | Chi² (RU²) | ka (1/Ms) | kd (1/s) | KD (M) | Rmax (RU) |
|---|---|---|---|---|---|---|
| NC | peptide-biotin | NA | NA | NA | NA | NA |
| VH + VL (A1) | peptide-biotin | 1.61E−02 | 3.25E+05 | 1.50E−03 | 4.61E−09 | 24.3 |
| VH1 + VL1 (A2) | peptide-biotin | 2.24E−02 | 2.49E+05 | 1.66E−03 | 6.66E−09 | 28.2 |
| VH1 + VL2 (A3) | peptide-biotin | 2.66E−01 | 3.27E+05 | 3.21E−03 | 9.81E−09 | 25.4 |
| VH1 + VL3 (A4) | peptide-biotin | 4.42E−02 | 2.52E+05 | 1.77E−03 | 7.00E−09 | 26.9 |
| VH1 + VL4 (A5) | peptide-biotin | 8.68E−02 | 2.17E+05 | 2.17E−03 | 9.96E−09 | 24 |
| VH2 + VL1 (A6) | peptide-biotin | 5.72E−02 | 1.84E+05 | 1.95E−03 | 1.06E−08 | 31.1 |
| VH2 + VL2 (A7) | peptide-biotin | 9.44E−02 | 2.56E+05 | 2.95E−03 | 1.15E−08 | 29.6 |
| VH2 + VL3 | peptide-biotin | 3.32E−02 | 2.03E+05 | 2.19E−03 | 1.08E−08 | 26.7 |
| VH2 + VL4 | peptide-biotin | 2.49E−02 | 2.15E+05 | 1.85E−03 | 8.62E−09 | 29.2 |
| VH3 + VL1 | peptide-biotin | 3.00E−02 | 3.42E+05 | 1.49E−03 | 4.36E−09 | 28.2 |
| VH3 + VL2 | peptide-biotin | 7.68E−02 | 4.38E+05 | 1.98E−03 | 4.53E−09 | 26.3 |
| VH3 + VL3 | peptide-biotin | 7.77E−02 | 4.09E+05 | 1.80E−03 | 4.40E−09 | 29.5 |
| VH3 + VL4 | peptide-biotin | 1.42E−01 | 6.08E+05 | 1.35E−03 | 2.22E−09 | 26.8 |
| VH4 + VL1 | peptide-biotin | 2.76E−02 | 3.05E+05 | 1.36E−03 | 4.47E−09 | 32.7 |
| VH4 + VL2 | peptide-biotin | 7.55E−02 | 3.03E+05 | 1.32E−03 | 4.37E−09 | 30.3 |
| VH4 + VL3 | peptide-biotin | 3.93E−02 | 3.32E+05 | 1.43E−03 | 4.32E−09 | 28.3 |
| VH4 + VL4 | peptide-biotin | 2.15E−01 | 4.54E+05 | 2.01E−03 | 4.43E−09 | 54.4 |

TABLE 2
Binding kinetics of antigen to antibodies

| Ligand | Analyte | Chi² (RU²) | ka (1/Ms) | kd (1/s) | KD (M) | Rmax (RU) |
|---|---|---|---|---|---|---|
| VH + VL (A1) | peptide-biotin | 1.24E−01 | 3.12E+05 | 5.43E−04 | 1.74E−09 | 44.2 |
| VH1 + VL1 (A2) | peptide-biotin | 1.04E−01 | 1.91E+05 | 6.05E−04 | 3.16E−09 | 45.8 |
| VH1 + VL3 (A4) | peptide-biotin | 7.35E−02 | 1.95E+05 | 6.20E−04 | 3.18E−09 | 46.1 |
| VH2 + VL1 (A6) | peptide-biotin | 1.62E−01 | 1.65E+05 | 6.62E−04 | 4.02E−09 | 48.3 |

TABLE 3
humanization design and back mutation sites

| | Chain | | | | | | | |
|---|---|---|---|---|---|---|---|---|
| Germline | Type | FR1 | CDR1 | FR2 | CDR2 | FR3 | CDR3 | FR4 |
| 72.4% | VH | QVQLQESPGLVKPSQSLSLTCTVT | GYSITSDYAWN | WIRQHPGNKLQWMG | YLSYSGVTSYNPSLK | RVTISRDTSKNQFSLKLSSVTAADTAVYYCAR | KGTYYRYEGSYWYFDV | WGAGTTVTVSS |
| IGHV4-31*02 | VH1 | QVQLQESPGLVKPSQSLSLTCTVS | GYSITSDYAWN | WIRQHPGKGLEWIG | YLSYSGVTSYNPSLK | RVTISRDTSKNQFSLKLSSVTAADTAVYYCAR | KGTYYRYEGSYWYFDV | WGQGTTVTVSS |
| | VH2 | QVQLQESPGLVKPSQSLSLTCTVS | GYSITSDYAWN | WIRQHPGKGLEWMG | YLSYSGVTSYNPSLK | RVTISRDTSKNQFSLKLSSVTAADTAVYYCAR | KGTYYRYEGSYWYFDV | WGQGTTVTVSS |
| | VH3 | QVQLQESPGLVKPSQSLSLTCTVS | GYSITSDYAWN | WIRQHPGKGLQWMG | YLSYSGVTSYNPSLK | RITISRDTSKNQFSLKLSSVTAADTAVYYCAR | KGTYYRYEGSYWYFDV | WGQGTTVTVSS |
| | VH4 | QVQLQESPGLVKPSQSLSLTCTVS | GYSITSDYAWN | WIRQHPGKGLQWMG | YLSYSGVTSYNPSLK | RITISRDTSKNQFELKLSSVTAADTAVYYCAR | KGTYYRYEGSYWYFDV | WGQGTTVTVSS |
| 77.9% | VH | DIQMNQSPSSLSASLGDTITITC | HANQNIDVWLS | WYQQKPGNVPKLLIY | KTSNLHT | GVPSRFGSGSGTDFTFTISSLQPEDIATYYC | QQGQSYPLT | FGGGTKLEIK |
| IGKV1-33*01 | VH1 | DIQMNQSPSSLSASVGDRVTITC | HANQNIDVWLS | WYQQKPGNVPKLLIY | KTSNLHT | GVPSRFGSGSGTDFTFTISSLQPEDIATYYC | QQGQSYPLT | FGGGTKLEIK |
| | VH2 | DIQMNQSPSSLSASVGDRVTITC | HANQNIDVWLS | WYQQKPGNVPKLLIY | KTSNLHT | GVPSRFGSGSGTDFTFTISSLQPEDIATYYC | QQGQSYPLT | FGGGTKLEIK |
| | VH3 | DIQMNQSPSSLSASVGDRVTITC | HANQNIDVWLS | WYQQKPGNVPKLLIY | KTSNLHT | GVPSRFGSGSGTDFTFTISSLQPEDIATYYC | QQGQSYPLT | FGGGTKLEIK |
| | VH4 | DIQMNQSPSSLSASVGDRVTITC | HANQNIDVWLS | WYQQKPGNVPKLLIY | KTSNLHT | GVPSRFGSGSGTDFTFTISSLQPEDIATYYC | QQGQSYPLT | FGGGTKLEIK |

### Stability of Humanized Antibodies

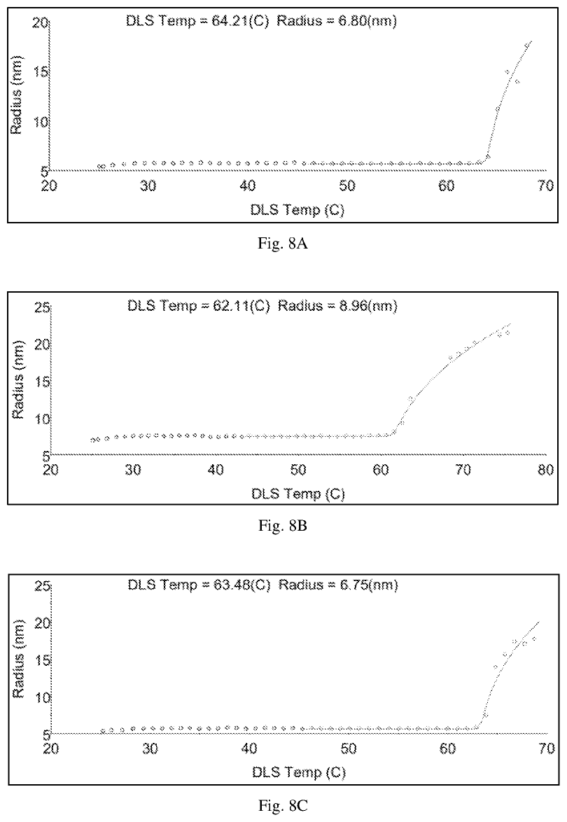

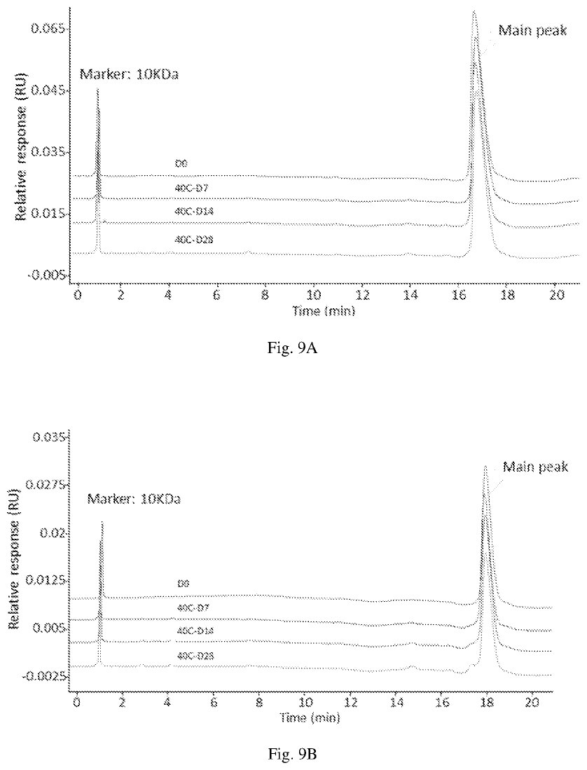

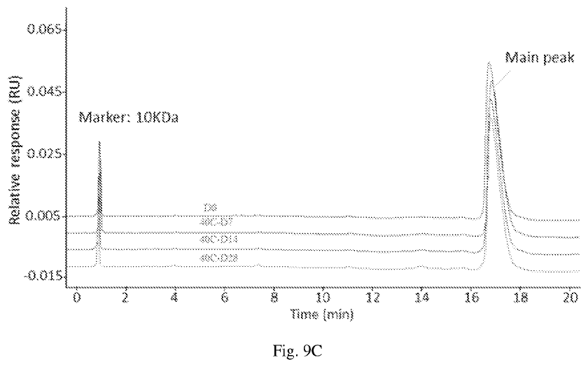

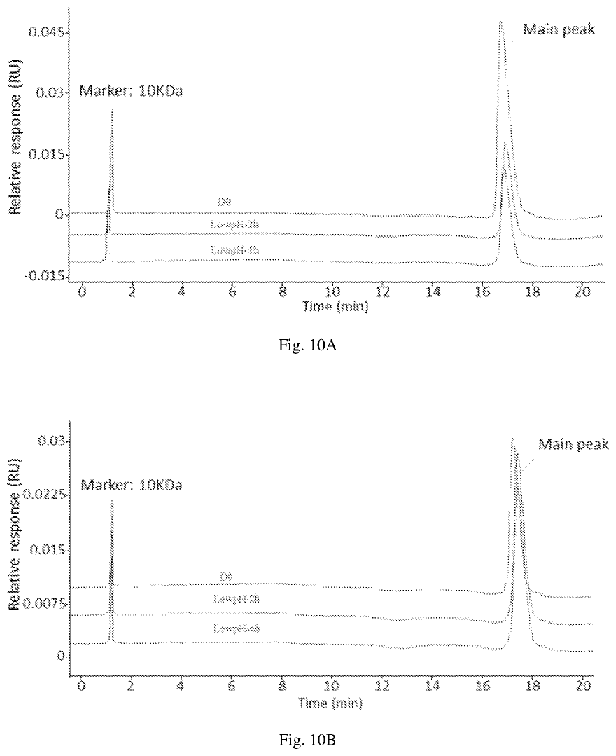

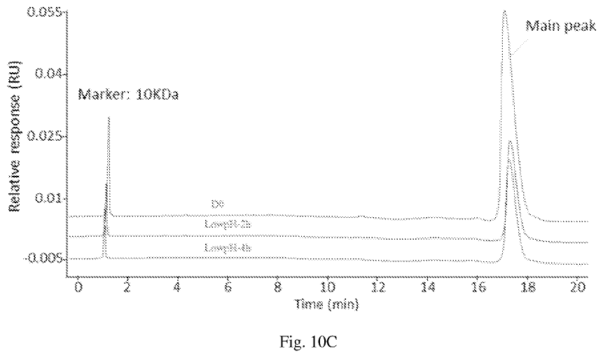

To determine the stability of the humanized antibodies, antibody clones A2, A4 and A6 were incubated under high temperature and low pH. The integrity of the antibody clones was then examined. All three antibody clones demonstrated well tolerance for high temperature (FIGS. 8A, 8B and 8C) and low pH (FIGS. 10A, 10B and 10C). The antibody clones were stable at above 60° C. (FIGS. 8A, 8B and 8C) and they demonstrated no signs of degradation under pH3.5 for up to 4 hours (FIGS. 10A, 10B and 10C). Antibody clones A2, A4 and A6 were also incubated at 40° C. for 28 days and all three antibody clones were stable after incubating for 28 days (FIGS. 9A, 9B and 9C). The high stability means that the humanized antibodies can maintain an active form in the body for a long time.

### Discussion

The present disclosure describes humanized antibodies specific against TRPM4 and their use in treating cytokine-associated vascular diseases. The humanized antibodies disclosed herein effectively inhibits TRPM4 activity.

Blocking TRPM4 provides a unique way of managing pulmonary edema and extrapulmonary vascular injury during lung infection and associated cytokine storm. TRPM4 expression is upregulated under hypoxia and LPS stimulation in vascular endothelial cells both from lung and extrapulmonary vasculature such as brain. It is known in the art that activation of TRPM4 by hypoxia and inflammation induce cell swelling and cell death. It is disclosed herein that TRPM4-blocking antibodies effectively protect pulmonary and cerebral vascular endothelial cells from hypoxia and inflammation induced cell death. In an animal model of pulmonary inflammation induced by LPS, addition of TRPM4-blocking antibody reduced inflammatory molecules production, ameliorated pulmonary injury with an improved survival rate. Therefore, it is apparent that TRPM4 blockers can serve as a broad vascular protector during pulmonary infection. With improved vascular protection, pulmonary edema can be reduced, and gas exchange in lungs can be improved. This is critical for improving the survival of patients with severe disease conditions. In addition, endotheliitis, an immune response within the endothelium in blood vessels, is found in organs such as brain, heart, liver, kidney, and small intestine such as during COVID-19 viral infection. Blocking TRPM4 can potentially alleviate the extrapulmonary injury during cytokine storm. Further, treatment with TRPM4 blocking antibodies is not limited to the treatment of viral infection, TRPM4 blocking antibodies can also be used to manage bacterial infection and fungal infection with pulmonary edema and associated cytokine storm.

The present disclosure describes for the first time:
1. The production and characterization of humanized antibody clones specific to TRPM4.
2. The therapeutic effects of the humanized antibodies against TRPM4 in treating cytokine-associated vascular disease.

The most important features of the present disclosure disclosed herein include:
1. The unique amino acid sequences of the variable domains for the heavy and light chains of the humanized antibody clones.
2. The broad therapeutic effects of the humanized antibodies in treating various types of cytokine-associated vascular diseases including those arising from viral, bacterial, and fungal infections.

The humanized antibodies of the present disclosure have the following advantages:
1. The humanized antibodies of the present disclosure are expected to demonstrate enhanced therapeutic effects especially in human patients due to lower risk of immune rejection compared to using existing rabbit or mouse antibodies specific against TRPM4.
2. The effective dosage of the humanized antibody clones required to inhibit TRPM4 activity in vitro is about 17 times lower than the effective dosage required for the mouse or rabbit antibody counterpart.

SEQUENCE LISTING

| SEQ | | |
|---|---|---|
| ID | | |
| NO | Sequence Name | Sequence |
| 1 | heavy chain CDR1 | GYSITSDYAWN |
| 2 | heavy chain CDR2 | YLSYSGVTSYNPSLKG |
| 3 | heavy chain CDR3 | KGTYYRYEGSYWYFDV |
| 4 | Light chain CDR1 | HANQNIDVWLS |
| 5 | Light chain CDR2 | KTSNLHT |
| 6 | Light chain CDR3 | QQGQSYPLT |
| 7 | VH1 variable region | QVQLQESGPGLVKPSQTLSLTCTVSGYSITSDYAWNWIRQHPG |
| | | KGLEWIGYLSYSGVTSYNPSLKGRVTISVDTSKNQFSLKLSSVT |
| | | AADTAVYYCARKGTYYRYEGSYWYFDVWGQGTTVTVSS |
| 8 | VH2 variable region | QVQLQESGPGLVKPSQTLSLTCTVSGYSITSDYAWNWIRQHPG |
| | | KGLEWMGYLSYSGVTSYNPSLKGRVTISRDTSKNQFSLKLSSV |
| | | TAADTAVYYCARKGTYYRYEGSYWYFDVWGQGTTVTVSS |
| 9 | VH3 variable region | QVQLQESGPGLVKPSQTLSLTCTVSGYSITSDYAWNWIRQHPG |
| | | KGLQWMGYLSYSGVTSYNPSLKGRITISRDTSKNQFSLKLSSV |
| | | TAADTAVYYCARKGTYYRYEGSYWYFDVWGQGTTVTVSS |
| 10 | VH4 variable region | QVQLQESGPGLVKPSQTLSLTCTVSGYSITSDYAWNWIRQHPG |
| | | KGLQWMGYLSYSGVTSYNPSLKGRITISRDTSKNQFFLKLSSV |
| | | TAADTAVYYCARKGTYYRYEGSYWYFDVWGQGTTVTVSS |
| 11 | VL1 variable region | DIQMTQSPSSLSASVGDRVTITCHANQNIDVWLSWYQQKPGKA |
| | | PKLLIYKTSNLHTGVPSRFSGSGSGTDFTFTISSLQPEDIATYYCQ |
| | | QGQSYPLTFGGGTKLEIK |
| 12 | VL2 variable region | DIQMTQSPSSLSASVGDRVTITCHANQNIDVWLSWYQQKPGKA |
| | | PKLLIYKTSNLHTGVPSRFSGSGSGTDFTLTISSLQPEDIATYYCQ |
| | | QGQSYPLTFGGGTKLEIK |
| 13 | VL3 variable region | DIQMTQSPSSLSASVGDRVTITCHANQNIDVWLSWYQQKPGNA |
| | | PKLLIYKTSNLHTGVPSRFSGSGSGTDFTFTISSLQPEDIATYYCQ |
| | | QGQSYPLTFGGGTKLEIK |
| 14 | VL4 variable region | DIQMTQSPSSLSASVGDRVTITCHANQNIDVWLSWYQQKPGNA |
| | | PKLLIYKTSNLHTGVPSRFSGSGSGTDFTLTISSLQPEDIATYYCQ |
| | | QGQSYPLTFGGGTKLEIK |
| 15 | TRPM4 Epitope | RDSDSNCSSEPGFWAHPPGAQ |
| 16 | TRPM4 Epitope | EPGF |
| 17 | Nucleotide sequence | ATGGGCTGGTCATGCATTATTCTGTTTCTGGTCGCAACTGCTAC |
| | encoding VH1 | AGGCGTGCATAGTCAAGTGCAGCTGCAGGAGAGCGGACCTGG |
| | | CCTGGTGAAGCCTTCTCAGACCCTGAGCCTCACCTGTACCGTG |
| | | TCCGGATATAGCATCACAAGCGACTACGCCTGGAATTGGATCA |
| | | GACAGCACCCAGGCAAGGGCCTGGAATGGATCGGCTACCTGTC |
| | | CTACAGCGGCGTGACATCTTATAACCCCAGCCTGAAAGGAAGA |
| | | GTGACCATCAGCGTGGACACCTCTAAGAACCAGTTCAGCCTGA |
| | | AGCTGTCTAGCGTCACAGCCGCTGATACCGCCGTGTACTACTGC |
| | | GCCAGAAAGGGCACCTACTACCGGTACGAGGGCAGCTACTGGT |
| | | ACTTCGACGTGTGGGGCCAGGGCACAACCGTGACCGTTTCCAG |
| | | CGCCAGCACCAAGGGCCCTTCCGTGTTTCCACTGGCCCCCTCCT |
| | | CTAAATCCACATCTGGCGGCACCGCCGCCCTGGGCTGTCTGGTG |
| | | AAGGACTACTTCCCAGAGCCTGTGACAGTGTCCTGGAACTCTGG |
| | | CGCCCTGACATCCGGCGTGCACACATTTCCAGCCGTGCTGCAGA |
| | | GCTCCGGCCTGTACAGCCTGTCTAGCGTGGTGACAGTGCCCTCC |
| | | TCTAGCCTGGGCACACAGACCTATATCTGCAACGTGAATCACAA |
| | | GCCAAGCAATACCAAGGTGGACAAGAAGGTGGAGCCCAAGTCC |
| | | TGTGATAAGACACACACCTGCCCCCCTTGTCCTGCTCCCGAGCT |
| | | GCTGGGCGGCCCTAGCGTGTTCCTGTTTCCACCCAAGCCTAAGG |
| | | ACACCCTGATGATCTCCCGGACACCCGAGGTGACCTGCGTGGT |
| | | GGTGGACGTGTCTCACGAGGATCCTGAGGTGAAGTTCAACTGG |
| | | TATGTGGATGGCGTGGAGGTGCACAATGCCAAGACCAAGCCCA |
| | | GAGAGGAGCAGTACAACTCTACATATAGGGTGGTGAGCGTGCT |
| | | GACCGTGCTGCACCAGGACTGGCTGAACGGCAAGGAGTATAAG |
| | | TGCAAGGTGTCCAATAAGGCCCTGCCCGCCCCCATCGAGAAGA |
| | | CAATCAGCAAGGCCAAGGGCCAGCCTCGGGAGCCACAGGTGTA |
| | | CACCCTGCCTCCATCCAGAGACGAGCTGACAAAGAACCAGGTG |
| | | TCTCTGACATGTCTGGTGAAGGGCTTCTATCCTAGCGATATCGC |
| | | CGTGGAGTGGGAGTCCAATGGCCAGCCAGAGAACAATTACAAG |
| | | ACCACACCCCCTGTGCTGGACTCCGATGGCTCCTTCTTTCTGTAT |
| | | TCCAAGCTGACCGTGGATAAGTCTCGGTGGCAGCAGGGCAACG |
| | | TGTTCAGCTGTTCCGTGATGCACGAAGCCCTGCATAATCACTAT |
| | | ACTCAGAAATCCCTGTCCCTGTCACCTGGAAAGTGATAA |
| 18 | Nucleotide sequence | ATGGGCTGGTCATGCATTATTCTGTTTCTGGTCGCAACTGCTAC |
| | encoding VH2 | AGGCGTGCATAGTCAAGTGCAGCTGCAGGAGAGCGGACCTGG |
| | | CCTGGTGAAGCCATCTCAGACCCTGTCTCTCACATGTACCGTG |
| | | TCTGGATATAGCATCACCAGCGACTACGCCTGGAACTGGATCC |
| | | GGCAGCACCCCGGCAAGGGCCTGGAATGGATGGGCTACCTGT |
| | | CCTACAGCGGAGTTACATCTTATAATCCTAGCCTGAAAGGCAG |
| | | AGTGACCATCAGCAGAGATACCTCCAAGAACCAGTTCAGCCT |
| | | GAAGCTGAGCAGCGTGACAGCCGCTGACACCGCCGTGTACTA |
| | | CTGCGCCAGAAAGGGCACATACTACCGGTACGAGGGCAGCTA |
| | | CTGGTACTTCGACGTGTGGGGCCAGGGCACCACCGTCACAGT |
| | | GTCCAGCGCCAGCACCAAGGGCCCTTCCGTGTTTCCACTGGC |
| | | CCCCTCCTCTAAATCCACATCTGGCGGCACCGCCGCCCTGGG |
| | | CTGTCTGGTGAAGGACTACTTCCCAGAGCCTGTGACAGTGTC |
| | | CTGGAACTCTGGCGCCCTGACATCCGGCGTGCACACATTTCC |
| | | AGCCGTGCTGCAGAGCTCCGGCCTGTACAGCCTGTCTAGCGT |
| | | GGTGACAGTGCCCTCCTCTAGCCTGGGCACACAGACCTATAT |
| | | CTGCAACGTGAATCACAAGCCAAGCAATACCAAGGTGGACA |
| | | AGAAGGTGGAGCCCAAGTCCTGTGATAAGACACACACCTGC |
| | | CCCCCTTGTCCTGCTCCCGAGCTGCTGGGCGGCCCTAGCGTG |
| | | TTCCTGTTTCCACCCAAGCCTAAGGACACCCTGATGATCTCC |
| | | CGGACACCCGAGGTGACCTGCGTGGTGGTGGACGTGTCTCA |
| | | CGAGGATCCTGAGGTGAAGTTCAACTGGTATGTGGATGGCG |
| | | TGGAGGTGCACAATGCCAAGACCAAGCCCAGAGAGGAGCA |
| | | GTACAACTCTACATATAGGGTGGTGAGCGTGCTGACCGTGC |
| | | TGCACCAGGACTGGCTGAACGGCAAGGAGTATAAGTGCAA |
| | | GGTGTCCAATAAGGCCCTGCCCGCCCCCATCGAGAAGACA |
| | | ATCAGCAAGGCCAAGGGCCAGCCTCGGGAGCCACAGGTGT |
| | | ACACCCTGCCTCCATCCAGAGACGAGCTGACAAAGAACCA |
| | | GGTGTCTCTGACATGTCTGGTGAAGGGCTTCTATCCTAGCG |
| | | ATATCGCCGTGGAGTGGGAGTCCAATGGCCAGCCAGAGAA |
| | | CAATTACAAGACCACACCCCCTGTGCTGGACTCCGATGGC |
| | | TCCTTCTTTCTGTATTCCAAGCTGACCGTGGATAAGTCTCG |
| | | GTGGCAGCAGGGCAACGTGTTCAGCTGTTCCGTGATGCAC |
| | | GAAGCCCTGCATAATCACTATACTCAGAAATCCCTGTCCCT |
| | | GTCACCTGGAAAGTGATAA |
| 19 | Nucleotide sequence | ATGGGCTGGTCATGCATTATTCTGTTTCTGGTCGCAACTGC |
| | encoding VH3 | TACAGGCGTGCATAGTCAAGTGCAGCTGCAGGAGTCTGGA |
| | | CCTGGACTGGTGAAGCCTAGCCAGACCCTGAGCCTGACCT |
| | | GTACCGTCTCCGGCTACAGCATCACCTCCGACTACGCCTGG |
| | | AATTGGATCAGACAGCACCCCGGCAAGGGCCTCCAGTGGA |
| | | TGGGCTACCTGTCTTATTCTGGAGTGACATCTTACAACCCA |
| | | AGCCTGAAAGGCAGAATCACCATCAGCCGGGACACCAGCA |
| | | AGAACCAGTTCAGCCTGAAGCTGAGCAGCGTGACCGCCGC |
| | | TGATACAGCCGTGTACTACTGCGCCAGAAAGGGCACCTAC |
| | | TACCGGTACGAAGGCAGCTATTGGTACTTCGACGTGTGGG |
| | | GCCAGGGCACAACAGTGACCGTGTCCAGCGCCAGCACCAA |
| | | GGGCCCTTCCGTGTTTCCACTGGCCCCCTCCTCTAAATCCA |
| | | CATCTGGCGGCACCGCCGCCCTGGGCTGTCTGGTGAAGGA |
| | | CTACTTCCCAGAGCCTGTGACAGTGTCCTGGAACTCTGGCG |
| | | CCCTGACATCCGGCGTGCACACATTTCCAGCCGTGCTGCAG |
| | | AGCTCCGGCCTGTACAGCCTGTCTAGCGTGGTGACAGTGCC |
| | | CTCCTCTAGCCTGGGCACACAGACCTATATCTGCAACGTGA |
| | | ATCACAAGCCAAGCAATACCAAGGTGGACAAGAAGGTGGA |
| | | GCCCAAGTCCTGTGATAAGACACACACCTGCCCCCCTTGTC |
| | | CTGCTCCCGAGCTGCTGGGCGGCCCTAGCGTGTTCCTGTTT |
| | | CCACCCAAGCCTAAGGACACCCTGATGATCTCCCGGACAC |
| | | CCGAGGTGACCTGCGTGGTGGTGGACGTGTCTCACGAGGA |
| | | TCCTGAGGTGAAGTTCAACTGGTATGTGGATGGCGTGGAG |
| | | GTGCACAATGCCAAGACCAAGCCCAGAGAGGAGCAGTAC |
| | | AACTCTACATATAGGGTGGTGAGCGTGCTGACCGTGCTGC |
| | | ACCAGGACTGGCTGAACGGCAAGGAGTATAAGTGCAAGG |
| | | TGTCCAATAAGGCCCTGCCCGCCCCCATCGAGAAGACAAT |
| | | CAGCAAGGCCAAGGGCCAGCCTCGGGAGCCACAGGTGTA |
| | | CACCCTGCCTCCATCCAGAGACGAGCTGACAAAGAACCA |
| | | GGTGTCTCTGACATGTCTGGTGAAGGGCTTCTATCCTAGC |
| | | GATATCGCCGTGGAGTGGGAGTCCAATGGCCAGCCAGAG |
| | | AACAATTACAAGACCACACCCCCTGTGCTGGACTCCGATG |
| | | GCTCCTTCTTTCTGTATTCCAAGCTGACCGTGGATAAGTCT |
| | | CGGTGGCAGCAGGGCAACGTGTTCAGCTGTTCCGTGATGC |
| | | ACGAAGCCCTGCATAATCACTATACTCAGAAATCCCTGTC |
| | | CCTGTCACCTGGAAAGTGATAA |
| 20 | Nucleotide sequence | ATGGGCTGGTCATGCATTATTCTGTTTCTGGTCGCAACTGCT |
| | encoding VH4 | ACAGGCGTGCATAGTCAAGTGCAGCTGCAGGAGAGCGGACC |
| | | TGGCCTGGTGAAGCCTAGCCAGACCCTGTCCCTGACATGTAC |
| | | CGTGTCTGGCTACAGCATCACAAGCGACTACGCCTGGAATTG |
| | | GATCCGGCAGCACCCAGGAAAGGGCCTCCAGTGGATGGGCT |
| | | ATCTGAGCTATAGCGGCGTGACATCCTACAACCCCAGCCTGA |
| | | AAGGAAGAATCACCATCAGCAGAGATACATCTAAGAACCAG |
| | | TTTTTCCTGAAGCTGTCTAGCGTGACCGCCGCTGACACCGCC |
| | | GTGTACTACTGCGCCAGAAAGGGCACCTACTACCGGTACGAA |
| | | GGCAGCTACTGGTACTTCGACGTGTGGGGCCAGGGCACAACC |
| | | GTCACCGTGTCCAGCGCCAGCACCAAGGGCCCTTCCGTGTTTC |
| | | CACTGGCCCCCTCCTCTAAATCCACATCTGGCGGCACCGCCGC |
| | | CCTGGGCTGTCTGGTGAAGGACTACTTCCCAGAGCCTGTGACA |
| | | GTGTCCTGGAACTCTGGCGCCCTGACATCCGGCGTGCACACAT |
| | | TTCCAGCCGTGCTGCAGAGCTCCGGCCTGTACAGCCTGTCTAG |
| | | CGTGGTGACAGTGCCCTCCTCTAGCCTGGGCACACAGACCTAT |
| | | ATCTGCAACGTGAATCACAAGCCAAGCAATACCAAGGTGGAC |
| | | AAGAAGGTGGAGCCCAAGTCCTGTGATAAGACACACACCTGC |
| | | CCCCCTTGTCCTGCTCCCGAGCTGCTGGGCGGCCCTAGCGTGT |
| | | TCCTGTTTCCACCCAAGCCTAAGGACACCCTGATGATCTCCCG |
| | | GACACCCGAGGTGACCTGCGTGGTGGTGGACGTGTCTCACGA |
| | | GGATCCTGAGGTGAAGTTCAACTGGTATGTGGATGGCGTGGA |
| | | GGTGCACAATGCCAAGACCAAGCCCAGAGAGGAGCAGTACA |
| | | ACTCTACATATAGGGTGGTGAGCGTGCTGACCGTGCTGCACC |
| | | AGGACTGGCTGAACGGCAAGGAGTATAAGTGCAAGGTGTCC |
| | | AATAAGGCCCTGCCCGCCCCCATCGAGAAGACAATCAGCAAG |
| | | GCCAAGGGCCAGCCTCGGGAGCCACAGGTGTACACCCTGCCT |
| | | CCATCCAGAGACGAGCTGACAAAGAACCAGGTGTCTCTGACA |
| | | TGTCTGGTGAAGGGCTTCTATCCTAGCGATATCGCCGTGGAGT |
| | | GGGAGTCCAATGGCCAGCCAGAGAACAATTACAAGACCACAC |
| | | CCCCTGTGCTGGACTCCGATGGCTCCTTCTTTCTGTATTCCAAG |
| | | CTGACCGTGGATAAGTCTCGGTGGCAGCAGGGCAACGTGTTCA |
| | | GCTGTTCCGTGATGCACGAAGCCCTGCATAATCACTATACTCA |
| | | GAAATCCCTGTCCCTGTCACCTGGAAAGTGATAA |
| 21 | Nucleotide sequence | ATGGGCTGGTCATGTATTATTCTGTTTCTGGTCGCAACTGCTAC |
| | encoding VL1 | AGGGGTCCATAGTGATATTCAGATGACCCAGAGCCCCAGCAGC |
| | | CTCTCCGCCAGCGTGGGAGATAGAGTGACCATCACCTGTCACG |
| | | CCAACCAGAATATCGACGTCTGGCTGAGCTGGTATCAGCAGAA |
| | | ACCTGGCAAGGCCCCTAAGCTGCTGATCTACAAGACCAGCAAC |
| | | CTGCACACCGGCGTGCCATCTCGGTTCAGCGGCTCTGGCAGCG |
| | | GCACCGACTTCACATTTACAATCAGCTCTCTGCAGCCTGAGGA |
| | | CATCGCTACATACTACTGCCAGCAAGGCCAGTCCTACCCCCTG |
| | | ACCTTCGGCGGAGGCACAAAGCTGGAAATCAAGAGGACAGTG |
| | | GCCGCCCCAAGCGTGTTCATCTTTCCCCCTTCCGACGAGCAGCT |
| | | GAAGTCTGGCACCGCCAGCGTGGTGTGCCTGCTGAACAACTTCT |
| | | ACCCTCGGGAGGCCAAGGTCCAGTGGAAGGTGGATAACGCCCT |
| | | GCAGTCTGGCAATAGCCAGGAGTCCGTGACCGAGCAGGACTCT |
| | | AAGGATAGCACATATTCCCTGTCTAGCACCCTGACACTGAGCA |
| | | AGGCCGATTACGAGAAGCACAAGGTGTATGCCTGTGAAGTCAC |
| | | CCATCAGGGGCTGTCATCACCCGTCACTAAGTCATTCAATCGCG |
| | | GAGAATGCTGATAA |
| 22 | Nucleotide sequence | ATGGGCTGGTCATGTATTATTCTGTTTCTGGTCGCAACTGCTAC |
| | encoding VL2 | AGGGGTCCATAGTGATATTCAGATGACCCAGTCCCCAAGCAGC |
| | | CTCTCCGCCAGCGTGGGCGATAGAGTGACCATCACCTGTCACG |
| | | CCAACCAGAATATCGACGTGTGGCTGAGCTGGTACCAGCAAAA |
| | | GCCCGGCAAAGCCCCTAAGCTGCTGATCTACAAGACCAGCAAC |
| | | CTGCACACCGGCGTCCCCAGCCGGTTCAGCGGATCTGGCAGCG |
| | | GCACCGACTTCACCCTGACAATCAGCTCTCTGCAGCCTGAGGA |
| | | CATCGCTACATACTACTGCCAGCAGGGCCAGTCTTATCCTCTGA |
| | | CATTTGGCGGCGGAACAAAGCTGGAAATCAAGAGGACAGTGGC |
| | | CGCCCCAAGCGTGTTCATCTTTCCCCCTTCCGACGAGCAGCTGA |
| | | AGTCTGGCACCGCCAGCGTGGTGTGCCTGCTGAACAACTTCTAC |
| | | CCTCGGGAGGCCAAGGTCCAGTGGAAGGTGGATAACGCCCTGC |
| | | AGTCTGGCAATAGCCAGGAGTCCGTGACCGAGCAGGACTCTAA |
| | | GGATAGCACATATTCCCTGTCTAGCACCCTGACACTGAGCAAG |
| | | GCCGATTACGAGAAGCACAAGGTGTATGCCTGTGAAGTCACCC |
| | | ATCAGGGGCTGTCATCACCCGTCACTAAGTCATTCAATCGCGG |
| | | AGAATGCTGATAA |
| 23 | Nucleotide sequence | ATGGGCTGGTCATGTATTATTCTGTTTCTGGTCGCAACTGCTAC |
| | encoding VL3 | AGGGGTCCATAGTGATATTCAGATGACCCAGAGCCCCAGCAG |
| | | CCTGTCCGCCAGCGTCGGCGATAGAGTGACAATCACCTGTCA |
| | | CGCCAACCAGAATATCGACGTGTGGCTGTCTTGGTATCAGCA |
| | | AAAACCTGGCAACGCTCCTAAGCTCCTGATCTACAAGACCAG |
| | | CAACCTGCACACAGGCGTGCCAAGCCGGTTCAGCGGCAGCGG |
| | | ATCTGGCACCGACTTCACCTTCACAATCAGCTCTCTGCAGCCTG |
| | | AGGACATCGCCACCTACTACTGCCAGCAGGGCCAGTCCTACCC |
| | | CCTGACCTTTGGCGGAGGCACAAAGCTGGAAATCAAGAGGACA |
| | | GTGGCCGCCCCAAGCGTGTTCATCTTTCCCCCTTCCGACGAGCA |
| | | GCTGAAGTCTGGCACCGCCAGCGTGGTGTGCCTGCTGAACAACT |
| | | TCTACCCTCGGGAGGCCAAGGTCCAGTGGAAGGTGGATAACGC |
| | | CCTGCAGTCTGGCAATAGCCAGGAGTCCGTGACCGAGCAGGAC |
| | | TCTAAGGATAGCACATATTCCCTGTCTAGCACCCTGACACTGAG |
| | | CAAGGCCGATTACGAGAAGCACAAGGTGTATGCCTGTGAAGTC |
| | | ACCCATCAGGGGCTGTCATCACCCGTCACTAAGTCATTCAATCG |
| | | CGGAGAATGCTGATAA |
| 24 | Nucleotide sequence | ATGGGCTGGTCATGTATTATTCTGTTTCTGGTCGCAACTGCTA |
| | encoding VL4 | CAGGGGTCCATAGTGATATTCAGATGACCCAGTCTCCATCTA |
| | | GCCTCTCCGCCAGCGTGGGCGACCGGGTGACCATCACCTGTCA |
| | | CGCCAACCAGAACATCGACGTGTGGCTGAGCTGGTATCAGCA |
| | | GAAACCTGGAAATGCCCCTAAGCTGCTGATCTACAAGACCAG |
| | | CAACCTGCACACCGGCGTCCCCAGCAGATTCAGCGGCAGCGG |
| | | CTCTGGCACCGACTTCACCCTGACAATCAGCAGCCTGCAGCCT |
| | | GAGGATATCGCTACATACTACTGCCAGCAAGGCCAGTCCTACC |
| | | CCCTGACATTTGGCGGCGGAACAAAGCTGGAAATCAAGAGGA |
| | | CAGTGGCCGCCCCAAGCGTGTTCATCTTTCCCCCTTCCGACGA |
| | | GCAGCTGAAGTCTGGCACCGCCAGCGTGGTGTGCCTGCTGAA |
| | | CAACTTCTACCCTCGGGAGGCCAAGGTCCAGTGGAAGGTGGA |
| | | TAACGCCCTGCAGTCTGGCAATAGCCAGGAGTCCGTGACCGA |
| | | GCAGGACTCTAAGGATAGCACATATTCCCTGTCTAGCACCCT |
| | | GACACTGAGCAAGGCCGATTACGAGAAGCACAAGGTGTATG |
| | | CCTGTGAAGTCACCCATCAGGGGCTGTCATCACCCGTCACTA |
| | | AGTCATTCAATCGCGGAGAATGCTGATAA |
| 25 | VH | QVQLQESGPGLVKPSQSLSLTCTVTGYSITSDYAWNWIRQFPGN |
| | | KLQWMGYLSYSGVTSYNPSLKGRISITRDTSKNQFFLQLSSVTP |
| | | EDTATYYCARKGTYYRYEGSYWYFDVWGAGTTVTVSS |
| 26 | VL | DIQMNQSPSSLSASLGDTITITCHANQNIDVWLSWYQQKPGNV |
| | | PKLLIYKTSNLHTGVPSRFSGSGSGTDFTLTISSLQPEDIATYYC |
| | | QQGQSYPLTFGGGTKLEIK |
| 27 | Nucleotide sequence | ATGGGCTGGTCATGCATTATTCTGTTTCTGGTCGCAACTGC |
| | encoding VH | TACAGGCGTGCATAGTCAGGTGCAGCTGCAGGAGAGCGGA |
| | | CCTGGCCTGGTGAAGCCTAGCCAGAGCCTGTCCCTCACCTG |
| | | TACCGTGACCGGCTACTCCATCACAAGCGACTACGCCTGGA |
| | | ATTGGATCAGACAGTTTCCAGGCAACAAGCTGCAGTGGATG |
| | | GGCTACCTGTCTTATTCTGGCGTGACATCTTACAACCCCAGC |
| | | CTGAAAGGCAGAATCAGCATCACCCGGGACACCAGCAAGAA |
| | | CCAGTTCTTCCTGCAACTGAGCAGCGTGACCCCTGAAGATA |
| | | CCGCCACATACTACTGCGCCAGAAAGGGCACCTACTACCGG |
| | | TACGAGGGCAGCTATTGGTACTTCGACGTGTGGGGCGCTGG |
| | | AACAACCGTCACAGTGTCCAGCGCCAGCACCAAGGGCCCTT |
| | | CCGTGTTTCCACTGGCCCCCTCCTCTAAATCCACATCTGGCG |
| | | GCACCGCCGCCCTGGGCTGTCTGGTGAAGGACTACTTCCCA |
| | | GAGCCTGTGACAGTGTCCTGGAACTCTGGCGCCCTGACATCC |
| | | GGCGTGCACACATTTCCAGCCGTGCTGCAGAGCTCCGGCCT |
| | | GTACAGCCTGTCTAGCGTGGTGACAGTGCCCTCCTCTAGCC |
| | | TGGGCACACAGACCTATATCTGCAACGTGAATCACAAGCC |
| | | AAGCAATACCAAGGTGGACAAGAAGGTGGAGCCCAAGTC |
| | | CTGTGATAAGACACACACCTGCCCCCCTTGTCCTGCTCCCG |
| | | AGCTGCTGGGCGGCCCTAGCGTGTTCCTGTTTCCACCCAAG |
| | | CCTAAGGACACCCTGATGATCTCCCGGACACCCGAGGTGA |
| | | CCTGCGTGGTGGTGGACGTGTCTCACGAGGATCCTGAGGT |
| | | GAAGTTCAACTGGTATGTGGATGGCGTGGAGGTGCACAAT |
| | | GCCAAGACCAAGCCCAGAGAGGAGCAGTACAACTCTACA |
| | | TATAGGGTGGTGAGCGTGCTGACCGTGCTGCACCAGGACT |
| | | GGCTGAACGGCAAGGAGTATAAGTGCAAGGTGTCCAATA |
| | | AGGCCCTGCCCGCCCCCATCGAGAAGACAATCAGCAAGG |
| | | CCAAGGGCCAGCCTCGGGAGCCACAGGTGTACACCCTGC |
| | | CTCCATCCAGAGACGAGCTGACAAAGAACCAGGTGTCTCT |
| | | GACATGTCTGGTGAAGGGCTTCTATCCTAGCGATATCGCC |
| | | GTGGAGTGGGAGTCCAATGGCCAGCCAGAGAACAATTAC |
| | | AAGACCACACCCCCTGTGCTGGACTCCGATGGCTCCTTCT |
| | | TTCTGTATTCCAAGCTGACCGTGGATAAGTCTCGGTGGCA |
| | | GCAGGGCAACGTGTTCAGCTGTTCCGTGATGCACGAAGC |
| | | CCTGCATAATCACTATACTCAGAAATCCCTGTCCCTGTCA |
| | | CCTGGAAAGTGATAA |
| 28 | Nucleotide sequence | ATGGGCTGGTCATGTATTATTCTGTTTCTGGTCGCAACTG |
| | encoding VL | CTACAGGGGTCCATAGTGATATCCAGATGAACCAGAGCC |
| | | CTTCTAGCCTCTCCGCCAGCCTGGGCGACACCATTACAAT |
| | | CACCTGTCACGCTAATCAGAACATCGACGTGTGGCTGAG |
| | | CTGGTATCAGCAGAAACCTGGCAACGTGCCCAAGCTGCT |
| | | GATCTACAAGACCAGCAACCTGCACACCGGCGTGCCATC |
| | | TAGATTCAGCGGATCTGGCAGCGGCACCGACTTCACCCTG |
| | | ACCATCAGCAGCCTGCAGCCTGAGGATATCGCCACATAC |
| | | TACTGCCAGCAAGGCCAGTCCTACCCCCTGACATTTGGCG |
| | | GCGGAACAAAGCTGGAAATCAAGAGGACAGTGGCCGCCC |
| | | CAAGCGTGTTCATCTTTCCCCCTTCCGACGAGCAGCTGAAG |
| | | TCTGGCACCGCCAGCGTGGTGTGCCTGCTGAACAACTTCT |
| | | ACCCTCGGGAGGCCAAGGTCCAGTGGAAGGTGGATAACG |
| | | CCCTGCAGTCTGGCAATAGCCAGGAGTCCGTGACCGAGC |
| | | AGGACTCTAAGGATAGCACATATTCCCTGTCTAGCACCC |
| | | TGACACTGAGCAAGGCCGATTACGAGAAGCACAAGGTG |
| | | TATGCCTGTGAAGTCACCCATCAGGGGCTGTCATCACCC |
| | | GTCACTAAGTCATTCAATCGCGGAGAATGCTGATAA |
| 29 | VH FR1 | QVQLQESGPGLVKPSQSLSLTCTVT |
| 30 | VH FR2 | WIRQFPGNKLQWMG |
| 31 | VH FR3 | RISITRDTSKNQFFLQLSSVTPEDTATYYCAR |
| 32 | VH FR4 | WGAGTTVTVSS |
| 33 | VH1/VH2/VH3/VH4 | QVQLQESGPGLVKPSQTLSLTCTVS |
| | FR1 | |
| 34 | VH1 FR2 | WIRQHPGKGLEWIG |
| 35 | VH1 FR3 | RVTISVDTSKNQFSLKLSSVTAADTAVYYCAR |
| 36 | VH1/VH2/VH3/VH4 | WGQGTTVTVSS |
| | FR4 | |
| 37 | VH2 FR2 | WIRQHPGKGLEWMG |
| 38 | VH2 FR3 | RVTISRDTSKNQFSLKLSSVTAADTAVYYCAR |
| 39 | VH3/VH4 FR2 | WIRQHPGKGLQWMG |
| 40 | VH3 FR3 | RITISRDTSKNQFSLKLSSVTAADTAVYYCAR |
| 41 | VH4 FR3 | RITISRDTSKNQFFLKLSSVTAADTAVYYCAR |
| 42 | VL FR1 | DIQMNQSPSSLSASLGDTITITC |
| 43 | VL FR2 | WYQQKPGNVPKLLIY |
| 44 | VL/VL2/VL4 FR3 | GVPSRFSGSGSGTDFTLTISSLQPEDIATYYC |
| 45 | Light chain FR4 | FGGGTKLEIK |
| 46 | VL1/VL2/VL3/VL4 | DIQMTQSPSSLSASVGDRVTITC |
| | FR1 | |
| 47 | VL1/VL2 FR2 | WYQQKPGKAPKLLIY |
| 48 | VL1/VL3 FR3 | GVPSRFSGSGSGTDFTFTISSLQPEDIATYYC |
| 49 | VL3/VL4 FR2 | WYQQKPGNAPKLLIY |
| 50 | VH1 | MGWSCIILFLVATATGVHSQVQLQESGPGLVKPSQTLSLT |
| | | CTVSGYSITSDYAWNWIRQHPGKGLEWIGYLSYSGVTSY |
| | | NPSLKGRVTISVDTSKNQFSLKLSSVTAADTAVYYCARK |
| | | GTYYRYEGSYWYFDVWGQGTTVTVSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSL |
| | | SPGK |
| 51 | VH2 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCAR |
| | | KGTYYRYEGSYWYFDVWGQGTTVTVSSASTKGPSVFPL |
| | | APSSKSTSGGTAALGCLVKDYFPEPVTVSWNSGALTSGV |
| | | HTFPAVLQSSGLYSLSSVVTVPSSSLGTQTYICNVNHKPS |
| | | NTKVDKKVEPKSCDKTHTCPPCPAPELLGGPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPSRDELTKNQ |
| | | VSLTCLVKGFYPSDIAVEWESNGQPENNYKTTPPVLDSDG |
| | | SFFLYSKLTVDKSRWQQGNVFSCSVMHEALHNHYTQKS |
| | | LSLSPGK |
| 52 | VH3 | MGWSCIILFLVATATGVHSQVQLQESGPGLVKPSQTLSLT |
| | | CTVSGYSITSDYAWNWIRQHPGKGLQWMGYLSYSGVTS |
| | | YNPSLKGRITISRDTSKNQFSLKLSSVTAADTAVYYCARK |
| | | GTYYRYEGSYWYFDVWGQGTTVTVSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSL |
| | | SPGK |
| 53 | VH4 | MGWSCIILFLVATATGVHSQVQLQESGPGLVKPSQTLSLT |
| | | CTVSGYSITSDYAWNWIRQHPGKGLQWMGYLSYSGVTS |
| | | YNPSLKGRITISRDTSKNQFFLKLSSVTAADTAVYYCARK |
| | | GTYYRYEGSYWYFDVWGQGTTVTVSSASTKGPSVFPLAP |
| | | SSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTK |
| | | PREEQYNSTYRVVSVLTVLHQDWLNGKEYKCKVSNKALP |
| | | APIEKTISKAKGQPREPQVYTLPPSRDELTKNQVSLTCLVK |
| | | GFYPSDIAVEWESNGQPENNYKTTPPVLDSDGSFFLYSKLT |
| | | VDKSRWQQGNVFSCSVMHEALHNHYTQKSLSLSPGK |
| 54 | VH5 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCARK |
| | | GTYYRYEGSYWYFDVWGQGTTVTVSSASTKGPSVFPLAP |
| | | SSKSTSGGTAALGCLVKDYFPEPVTVSWNSGALTSGVHTF |
| | | PAVLQSSGLYSLSSVVTVPSSSLGTQTYICNVNHKPSNTKV |
| | | DKKVEPKSCDKTHTCPPCPAPEAAGGPSVFLFPPKPKDTL |
| | | MISRTPEVTCVVVDVSHEDPEVKFNWYVDGVEVHNAKT |
| | | KPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLY |
| | | SKLTVDKSRWQQGNVFSCSVMHEALHNHYTQKSLSLSPGK |
| 55 | VH6 | MGWSCIILFLVATATGVHSQVQLQESGPGLVKPSQTLSLT |
| | | CTVSGYSITSDYAWNWIRQHPGKGLEWMGYLSYSGVTSY |
| | | NPSLKGRVTISRDTSKNQFSLKLSSVTAADTAVYYCARKG |
| | | TYYRYEGSYWYFDVWGQGTTVTVSSASTKGPSVFPLAPS |
| | | SKSTSGGTAALGCLVKDYFPEPVTVSWNSGALTSGVHTFP |
| | | AVLQSSGLYSLSSVVTVPSSSLGTQTYICNVNHKPSNTKVD |
| | | KKVEPKSCDKTHTCPPCPAPELLGGPSVFLFPPKPKDTLMI |
| | | SRTPEVTCVVVDVSHEDPEVKFNWYVDGVEVHNAKTKPR |
| | | EEQYASTYRVVSVLTVLHQDWLNGKEYKCKVSNKALPAPI |
| | | EKTISKAKGQPREPQVYTLPPSRDELTKNQVSLTCLVKGFY |
| | | PSDIAVEWESNGQPENNYKTTPPVLDSDGSFFLYSKLTVDK |
| | | SRWQQGNVFSCSVMHEALHNHYTQKSLSLSPGK |
| 56 | VH7 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCARK |
| | | GTYYRYEGSYWYFDVWGQGTTVTVSSASTKGPSVFPLAP |
| | | CSRSTSESTAALGCLVKDYFPEPVTVSWNSGALTSGVHTF |
| | | PAVLQSSGLYSLSSVVTVPSSSLGTKTYTCNVDHKPSNTK |
| | | VDKRVESKYGPPCPPCPAPEFLGGPSVFLFPPKPKDTLMIS |
| | | RTPEVTCVVVDVSQEDPEVQFNWYVDGVEVHNAKTKPR |
| | | EEQFNSTYRVVSVLTVLHQDWLNGKEYKCKVSNKGLPSS |
| | | IEKTISKAKGQPREPQVYTLPPSQEEMTKNQVSLTCLVKG |
| | | FYPSDIAVEWESNGQPENNYKTTPPVLDSDGSFFLYSRLT |
| | | VDKSRWQEGNVFSCSVMHEALHNHYTQKSLSLSLGK |
| 57 | VL1 | MGWSCIILFLVATATGVHSDIQMTQSPSSLSASVGDRVTI |
| | | TCHANQNIDVWLSWYQQKPGKAPKLLIYKTSNLHTGVP |
| | | SRFSGSGSGTDFTFTISSLQPEDIATYYCQQGQSYPLTFGG |
| | | GTKLEIKRTVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 58 | VL2 | MGWSCIILFLVATATGVHSDIQMTQSPSSLSASVGDRVTI |
| | | TCHANQNIDVWLSWYQQKPGKAPKLLIYKTSNLHTGVP |
| | | SRFSGSGSGTDFTLTISSLQPEDIATYYCQQGQSYPLTFGG |
| | | GTKLEIKRTVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 59 | VL3 | MGWSCIILFLVATATGVHSDIQMTQSPSSLSASVGDRVTI |
| | | TCHANQNIDVWLSWYQQKPGNAPKLLIYKTSNLHTGVP |
| | | SRFSGSGSGTDFTFTISSLQPEDIATYYCQQGQSYPLTFGG |
| | | GTKLEIKRTVAAPSVFIFPPSDEQLKSGTASVVCLLNNFY |
| | | PREAKVQWKVDNALQSGNSQESVTEQDSKDSTYSLSST |
| | | LTLSKADYEKHKVYACEVTHQGLSSPVTKSFNRGEC |
| 60 | VL4 | MGWSCIILFLVATATGVHSDIQMTQSPSSLSASVGDRVTI |
| | | TCHANQNIDVWLSWYQQKPGNAPKLLIYKTSNLHTGVP |
| | | SRFSGSGSGTDFTLTISSLQPEDIATYYCQQGQSYPLTFG |
| | | GGTKLEIKRTVAAPSVFIFPPSDEQLKSGTASVVCLLNNF |
| | | YPREAKVQWKVDNALQSGNSQESVTEQDSKDSTYSLSS |
| | | TLTLSKADYEKHKVYACEVTHQGLSSPVTKSFNRGEC |
| 61 | Nucleotide sequence | ATGGGCTGGTCATGCATTATTCTGTTTCTGGTCGCAAC |
| | encoding VH5 | TGCTACAGGCGTGCATAGTCAAGTGCAGCTGCAGGAG |
| | | AGCGGACCTGGCCTGGTGAAGCCATCTCAGACCCTGT |
| | | CTCTCACATGTACCGTGTCTGGATATAGCATCACCAGC |
| | | GACTACGCCTGGAACTGGATCCGGCAGCACCCCGGCA |
| | | AGGGCCTGGAATGGATGGGCTACCTGTCCTACAGCGG |
| | | AGTTACATCTTATAATCCTAGCCTGAAAGGCAGAGTGA |
| | | CCATCAGCAGAGATACCTCCAAGAACCAGTTCAGCCT |
| | | GAAGCTGAGCAGCGTGACAGCCGCTGACACCGCCGTG |
| | | TACTACTGCGCCAGAAAGGGCACATACTACCGGTACGA |
| | | GGGCAGCTACTGGTACTTCGACGTGTGGGGCCAGGGCA |
| | | CCACCGTCACAGTGTCCAGCGCCAGCACAAAGGGCCC |
| | | TAGCGTGTTTCCACTGGCCCCCTCCTCTAAGTCCACCT |
| | | CTGGAGGAACAGCCGCCCTGGGCTGTCTGGTGAAGGAT |
| | | TATTTCCCAGAGCCCGTGACCGTGTCCTGGAACTCTGGC |
| | | GCCCTGACCAGCGGAGTGCACACATTTCCAGCCGTGCTG |
| | | CAGAGCTCCGGACTGTACTCCCTGTCTAGCGTGGTGACC |
| | | GTGCCTTCCTCTAGCCTGGGCACCCAGACATATATCTGC |
| | | AACGTGAATCACAAGCCCTCCAATACAAAGGTGGACAA |
| | | GAAGGTGGAGCCTAAGTCTTGTGATAAGACCCACACATG |
| | | CCCCCCTTGTCCTGCACCAGAGGCAGCAGGAGGACCTT |
| | | CCGTGTTCCTGTTTCCACCCAAGCCAAAGGACACCCTG |
| | | ATGATCAGCCGCACCCCTGAGGTGACATGCGTGGTGGT |
| | | GGACGTGTCCCACGAGGATCCAGAGGTGAAGTTTAACT |
| | | GGTACGTGGATGGCGTGGAGGTGCACAATGCCAAGACC |
| | | AAGCCTCGGGAGGAGCAGTACAACTCTACCTATAGAGTG |
| | | GTGAGCGTGCTGACAGTGCTGCACCAGGACTGGCTGAAC |
| | | GGCAAGGAGTATAAGTGCAAGGTGTCTAATAAGGCCCT |
| | | GCCCGCCCCTATCGAGAAGACCATCAGCAAGGCAAAGG |
| | | GACAGCCTAGGGAGCCACAGGTGTACACACTGCCTCCA |
| | | TCTAGAGACGAGCTGACCAAGAACCAGGTGAGCCTGACA |
| | | TGTCTGGTGAAGGGCTTCTATCCAAGCGATATCGCCGTG |
| | | GAGTGGGAGTCCAATGGCCAGCCCGAGAACAATTACAAG |
| | | ACCACACCCCCTGTGCTGGACAGCGATGGCTCCTTCTTTC |
| | | TGTATTCCAAGCTGACCGTGGATAAGTCTCGGTGGCAGC |
| | | AGGGCAACGTGTTTAGCTGTTCCGTGATGCACGAGGCCC |
| | | TGCACAATCACTACACCCAGAAGTCTCTGAGCCTGTCC |
| | | CCCGGCAAGTGA |
| 62 | Nucleotide sequence | ATGGGCTGGTCATGCATTATTCTGTTTCTGGTCGCAAC |
| | encoding VH6 | TGCTACAGGCGTGCATAGTCAAGTGCAGCTGCAGGAGA |
| | | GCGGACCTGGCCTGGTGAAGCCATCTCAGACCCTGTCT |
| | | CTCACATGTACCGTGTCTGGATATAGCATCACCAGCGAC |
| | | TACGCCTGGAACTGGATCCGGCAGCACCCCGGCAAGGG |
| | | CCTGGAATGGATGGGCTACCTGTCCTACAGCGGAGTTA |
| | | CATCTTATAATCCTAGCCTGAAAGGCAGAGTGACCATC |
| | | AGCAGAGATACCTCCAAGAACCAGTTCAGCCTGAAGCT |
| | | GAGCAGCGTGACAGCCGCTGACACCGCCGTGTACTACT |
| | | GCGCCAGAAAGGGCACATACTACCGGTACGAGGGCAG |
| | | CTACTGGTACTTCGACGTGTGGGGCCAGGGCACCACCG |
| | | TCACAGTGTCCAGCGCCAGCACCAAGGGCCCTTCCGTG |
| | | TTTCCACTGGCCCCCTCCTCTAAATCCACATCTGGCGGC |
| | | ACCGCCGCCCTGGGCTGTCTGGTGAAGGACTACTTCCC |
| | | AGAGCCTGTGACAGTGTCCTGGAACTCTGGCGCCCTGA |
| | | CATCCGGCGTGCACACATTTCCAGCCGTGCTGCAGAGCT |
| | | CCGGCCTGTACAGCCTGTCTAGCGTGGTGACAGTGCCC |
| | | TCCTCTAGCCTGGGCACACAGACCTATATCTGCAACGT |
| | | GAATCACAAGCCAAGCAATACCAAGGTGGACAAGAAG |
| | | GTGGAGCCCAAGTCCTGTGATAAGACACACACCTGCCC |
| | | CCCTTGTCCTGCTCCCGAGCTGCTGGGCGGCCCTAGCG |
| | | TGTTCCTGTTTCCACCCAAGCCTAAGGACACCCTGATG |
| | | ATCTCCCGGACACCCGAGGTGACCTGCGTGGTGGTGGA |
| | | CGTGTCTCACGAGGATCCTGAGGTGAAGTTCAACTGGT |
| | | ATGTGGATGGCGTGGAGGTGCACAATGCCAAGACCAA |
| | | GCCCAGAGAGGAGCAGTACGCCTCTACATATAGGGTG |
| | | GTGAGCGTGCTGACCGTGCTGCACCAGGACTGGCTGAA |
| | | CGGCAAGGAGTATAAGTGCAAGGTGTCCAATAAGGCC |
| | | CTGCCCGCCCCCATCGAGAAGACAATCAGCAAGGCCA |
| | | AGGGCCAGCCTCGGGAGCCACAGGTGTACACCCTGCCT |
| | | CCATCCAGAGACGAGCTGACAAAGAACCAGGTGTCTC |
| | | TGACATGTCTGGTGAAGGGCTTCTATCCTAGCGATATC |
| | | GCCGTGGAGTGGGAGTCCAATGGCCAGCCAGAGAACA |
| | | ATTACAAGACCACACCCCCTGTGCTGGACTCCGATGGC |
| | | TCCTTCTTTCTGTATTCCAAGCTGACCGTGGATAAGTCT |
| | | CGGTGGCAGCAGGGCAACGTGTTCAGCTGTTCCGTGAT |
| | | GCACGAAGCCCTGCATAATCACTATACTCAGAAATCCC |
| | | TGTCCCTGTCACCTGGAAAGTGA |
| 63 | Nucleotide sequence | ATGGGCTGGTCTTGTATTATTCTGTTTCTGGTCGCAAC |
| | encoding VH7 | TGCTACAGGCGTGCATTCTCAAGTGCAGCTGCAGGAG |
| | | AGCGGACCTGGCCTGGTGAAGCCATCTCAGACCCTGT |
| | | CTCTCACATGTACCGTGTCTGGATATAGCATCACCAGC |
| | | GACTACGCCTGGAACTGGATCCGGCAGCACCCCGGCA |
| | | AGGGCCTGGAATGGATGGGCTACCTGTCCTACAGCGG |
| | | AGTTACATCTTATAATCCTAGCCTGAAAGGCAGAGTG |
| | | ACCATCAGCAGAGATACCTCCAAGAACCAGTTCAGCC |
| | | TGAAGCTGAGCAGCGTGACAGCCGCTGACACCGCCGT |
| | | GTACTACTGCGCCAGAAAGGGCACATACTACCGGTAC |
| | | GAGGGCAGCTACTGGTACTTCGACGTGTGGGGCCAGG |
| | | GCACCACCGTCACAGTGTCCAGCGCCAGCACCAAGGG |
| | | CCCTTCCGTGTTTCCCCTGGCCCCTTGCTCCCGGTCCAC |
| | | ATCTGAGAGCACCGCCGCCCTGGGCTGTCTGGTGAAG |
| | | GACTACTTCCCAGAGCCCGTGACCGTGAGCTGGAACA |
| | | GCGGCGCCCTGACAAGCGGCGTGCACACATTTCCCGC |
| | | CGTGCTGCAGAGCTCCGGCCTGTACTCCCTGTCTAGCG |
| | | TGGTGACAGTGCCTTCCTCTAGCCTGGGCACCAAGACA |
| | | TATACCTGTAACGTGGACCACAAGCCAAGCAATACCA |
| | | AGGTGGATAAGCGGGTGGAGTCTAAGTACGGCCCTCC |
| | | TTGCCCTCCATGTCCTGCTCCAGAGTTTCTGGGCGGCC |
| | | CTTCCGTGTTCCTGTTTCCACCCAAACCAAAGGACACA |
| | | CTGATGATCTCTAGAACACCAGAGGTGACCTGCGTGGT |
| | | GGTGGACGTGAGCCAGGAGGATCCCGAGGTGCAGTTC |
| | | AACTGGTACGTGGATGGCGTGGAGGTGCACAATGCCA |
| | | AGACCAAGCCAAGAGAGGAGCAGTTTAACTCTACATA |
| | | CAGGGTGGTGAGCGTGCTGACCGTGCTGCACCAGGAT |
| | | TGGCTCAACGGCAAGGAGTATAAGTGCAAGGTGTCCA |
| | | ATAAGGGCCTGCCCTCCTCTATCGAGAAGACAATCTCT |
| | | AAGGCTAAGGGCCAGCCAAGAGAGCCTCAGGTGTACA |
| | | CCCTGCCTCCAAGCCAGGAGGAGATGACAAAGAACCA |
| | | GGTGTCCCTGACATGTCTGGTGAAGGGCTTCTATCCCT |
| | | CCGACATCGCCGTGGAGTGGGAGTCTAATGGCCAGCC |
| | | TGAGAACAATTACAAGACCACACCCCCTGTGCTGGAC |
| | | TCTGATGGCAGCTTCTTTCTGTATTCCAGGCTGACCGT |
| | | GGATAAGTCTCGGTGGCAGGAGGGCAACGTGTTCAGC |
| | | TGCTCTGTGATGCACGAAGCCCTGCATAATCACTATA |
| | | CTCAGAAAAGTCTGTCACTGTCACTGGGAAAGTGA |

What is claimed is:

1. A humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

2. The antibody or antigen-binding fragment thereof of claim 1, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14.

3. The antibody or antigen-binding fragment thereof of claim 1, wherein:
a) the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11;
b) the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12;
c) the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 13;
d) the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 14;
e) the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11; or
f) the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

4. The antibody or antigen-binding fragment thereof of claim 1, wherein the antibody or antigen-binding fragment thereof comprises:
(a) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56; or
(b) a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

5. (canceled)

6. The antibody or antigen-binding fragment thereof of claim 1, wherein the antibody:
(a) specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16; or
(b) inhibits TRPM4 activity; or
(c) inhibits TRPM4 current and/or internalizes membrane TRPM4 protein.

7. (canceled)

8. (canceled)

9. A nucleic acid encoding the antibody or antigen-binding fragment thereof of claim 1.

10. The nucleic acid of claim 9, wherein the nucleic acid comprises a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63.

11. An expression vector comprising the nucleic acid of claim 9.

12. A host cell comprising the nucleic acid of claim 9.

13. A method of producing the antibody or antigen-binding fragment thereof of claim 1, the method comprising: (i) culturing a host cell comprising a nucleic acid encoding the antibody or antigen-binding fragment thereof in a culture medium, and (ii) isolating the antibody or antigen-binding fragment thereof from the culture medium.

14. (canceled)

15. A pharmaceutical composition comprising the antibody or antigen-binding fragment thereof of claim 1, and a pharmaceutically acceptable carrier, excipient, or diluent.

16. (canceled)

17. A method of treating cytokine-associated vascular disease, comprising administering to a subject an effective amount of the antibody or antigen-binding fragment thereof of claim 1.

18. (canceled)

19. The method of claim 17, wherein the effective amount of the antibody or antigen-binding fragment thereof;
(a) is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg; or
(b) treats cytokine-associated vascular disease by preventing vascular damage in the subject, wherein optionally the vascular damage is prevented by preventing hypoxia and inflammation-induced cell death of vascular cells, and wherein optionally, the vascular cells are pulmonary and cerebral vascular endothelial cells.

20. (canceled)

21. The method of claim 17, wherein the cytokine-associated vascular disease is selected from the group consisting of pulmonary edema, extrapulmonary vascular injury, pulmonary inflammation, lung infection, viral infection associated with pulmonary edema, bacterial infection associated with pulmonary edema, fungal infection associated with pulmonary edema, wherein optionally the cytokine-associated vascular disease is pulmonary inflammation.

22. The method of claim 21, wherein;
the viral infection associated with pulmonary edema is associated to a virus selected from the group consisting of cytomegalovirus, Epstein-Barr virus, influenza virus, variola virus and severe acute respiratory syndrome coronavirus (SARS-CoV), wherein optionally the virus is SARS-CoV;
the bacterial infection associated with pulmonary edema is associated to a bacteria selected from the group consisting of group A streptococcus, Haemophilus bacteria, Staphylococcus aureus and Mycobacterium tuberculosis; and
the fungal infection associated with pulmonary edema is associated to a fungus selected from the group consisting of Aspergillus, Cryptococcus, Pneumocystis and endemic fungi, wherein the endemic fungus is optionally selected from the group consisting of Sporothrix schenckii, Coccidioides immitis and Coccidioides posadasii, Blastomyces dermatitidis and Histoplasma capsulatum.

23. (canceled)

24. (canceled)

25. A kit for treating cytokine-associated vascular disease comprising the antibody or antigen-binding fragment thereof of claim 1 and/or instructions for use.

26. A host cell comprising the expression vector of claim 11.

27. A method of treating cytokine-associated vascular disease, comprising administering to a subject an effective amount of the pharmaceutical composition of claim 15.

28. The method of claim 27, wherein the effective amount of the antibody or antigen-binding fragment thereof:
(a) is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg; or
(b) treats cytokine-associated vascular disease by preventing vascular damage in the subject, wherein optionally the vascular damage is prevented by preventing hypoxia and inflammation-induced cell death of vascular cells, and wherein optionally, the vascular cells are pulmonary and cerebral vascular endothelial cells.

29. The method of claim 27, wherein the cytokine-associated vascular disease is selected from the group consisting of pulmonary edema, extrapulmonary vascular injury, pulmonary inflammation, lung infection, viral infection associated with pulmonary edema, bacterial infection associated with pulmonary edema, fungal infection associated with pulmonary edema, wherein optionally the cytokine-associated vascular disease is pulmonary inflammation.

30. The method of claim 29, wherein:
the viral infection associated with pulmonary edema is associated to a virus selected from the group consisting of cytomegalovirus, Epstein-Barr virus, influenza virus, variola virus and severe acute respiratory syndrome coronavirus (SARS-CoV), wherein optionally the virus is SARS-CoV;
the bacterial infection associated with pulmonary edema is associated to a bacteria selected from the group consisting of group A streptococcus, Haemophilus bacteria, Staphylococcus aureus and Mycobacterium tuberculosis; and
the fungal infection associated with pulmonary edema is associated to a fungus selected from the group consisting of Aspergillus, Cryptococcus, Pneumocystis and endemic fungi, wherein the endemic fungus is optionally selected from the group consisting of Sporothrix schenckii, Coccidioides immitis and Coccidioides posadasii, Blastomyces dermatitidis and Histoplasma capsulatum.
